(19) United States
(12) Patent Application Publication
(10) Pub. No.: US 2013/0189684 A1
(43) Pub. Date: Jul. 25, 2013

(54) QUANTIFICATION OF CELL-SPECIFIC NUCLEIC ACID MARKERS

(71) Applicant: SEQUENOM, INC., San Diego, CA (US)

(72) Inventors: Mathias EHRICH, San Diego, CA (US); Taylor JENSEN, San Diego, CA (US)

(73) Assignee: SEQUENOM, INC., San Diego, CA (US)

(21) Appl. No.: 13/797,377

(22) Filed: Mar. 12, 2013

(51) Int. Cl. C12Q 1/68

(52) U.S. Cl. CPC C12Q 1/6883; C12Q 1/6886
USPC 435/6.11

(57) ABSTRACT

The technology relates in part to selection, quantification and use of particular nucleic acid markers. In some embodiments, such markers are particular epigenetic markers, and sometimes each marker is a particular methylation state of a nucleic acid locus.

### FIELD

The technology relates in part to selection, quantification and use of particular nucleic acid markers. In some embodiments, such markers are epigenetic markers, and sometimes each marker is a particular methylation state of a nucleic acid locus.

### BACKGROUND

A marker in nucleic acid sometimes includes one or more bases that can be modified by an epigenetic modification, and a marker sometimes is a particular methylation state for a subset of nucleotides in a nucleic acid (i.e., a nucleic acid locus). A methylation state generally describes one or more characteristics of a nucleic acid at a particular locus relevant to methylation. Such characteristics include, but are not limited to, whether any of the cytosine (C) bases within a locus are methylated, location of methylated C base(s), percentage of methylated C base(s) at a particular locus, and allelic differences in methylation due to, for example, difference in the origin of alleles. A methylation state sometimes is a relative or absolute amount of methylated C or non-methylated C at a particular locus in a nucleic acid. Detecting a methylation state or a change in methylation state may be utilized for assessing the state of a cell or tissue or make a diagnostic determination, for example.

### SUMMARY

Provided in certain aspects is a method for quantifying one or more nucleic acid markers, including: (a) exposing circulating cell-free nucleic acid to conditions that permit quantification of the amount of one or more markers in the nucleic acid, wherein: each of the one or more markers is a particular methylation state of a region of the nucleic acid, and the methylation state of each of the one or more markers is the same or substantially the same for a cell type in subjects having a medical condition and for the cell type in subjects not having the medical condition; and (b) quantifying the amount of each of the one or more markers in the nucleic acid, thereby providing a quantification of the one or more markers, with the proviso that the presence or absence of a change in the methylation state of the one or more markers is not determined.

Also provided in certain aspects is a method for preparing a collection of nucleic acid markers, including: (a) determining the methylation state of multiple loci in nucleic acid from multiple cell types from multiple subjects; and (b) selecting loci for which the methylation state is the same or substantially the same for a cell type in subjects having a medical condition and for the cell type in subjects not having the medical condition; whereby a collection of nucleic acid markers is prepared. Such a method sometimes includes synthesizing one or more loci in the collection of markers, and sometimes the synthesizing includes amplifying a portion of nucleic acid from a subject including one of the loci, or amplifying portions of nucleic acid including a plurality of loci.

Provided also in some aspects is a method for obtaining a collection of amplification primers, including: (a) determining the methylation state of multiple loci in nucleic acid from multiple cell types from multiple subjects; (b) selecting loci for which the methylation state is the same or substantially the same for a cell type in subjects having a medical condition and for the cell type in subjects not having the medical condition; and (c) designing amplification primers, each of which primers is capable of amplifying each of the loci selected in (b); whereby a collection of amplification primers is obtained. Such a method sometimes includes synthesizing the collection of amplification primers.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows use of a distinguishing feature of cell types, in this case DNA methylation. The difference shown is not between diseased and non-diseased variants of the same cell type, but rather between two distinct cell types.

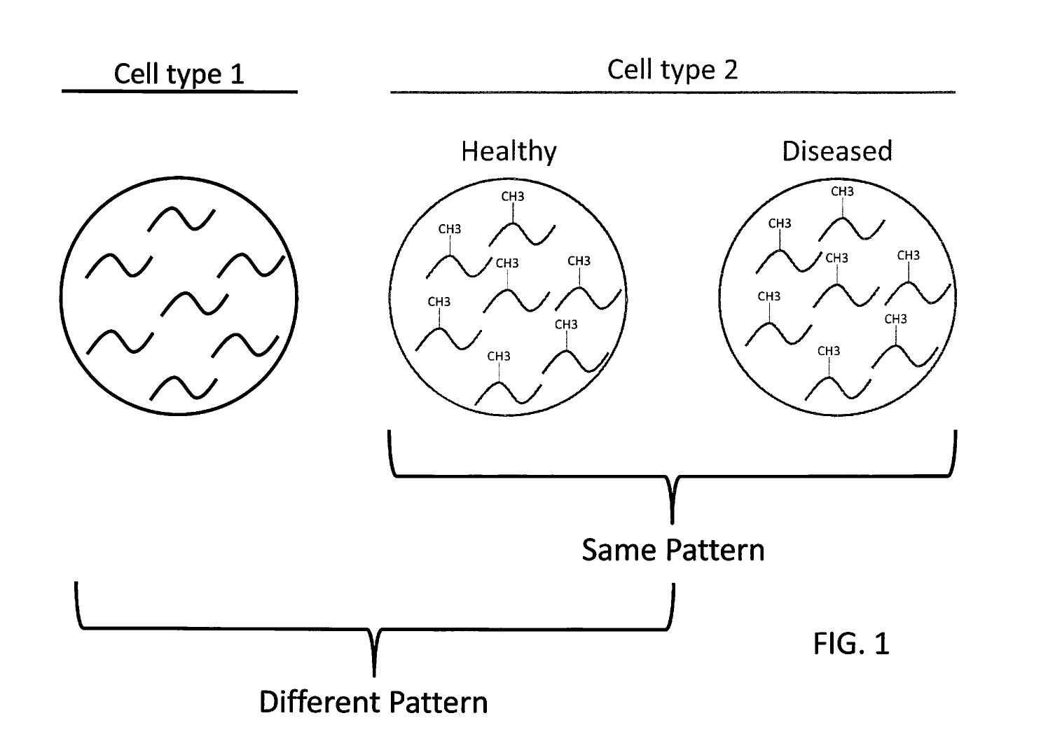

FIG. 2 shows a schematic of the use of cell type specific differentiation, using DNA methylation as an example. Shown are the absolute or relative quantity of nucleic acid contributed from cell type 1 (black) or cell type 2 (gray) in a healthy individual. Also shown are the absolute or relative increase that would be observed in the case of a condition resulting in an absolute or relative increase in nucleic acids from cell type 2 (Diseased-Gain) or a condition resulting in an absolute or relative decrease in nucleic acids from cell type 2 (Diseased-Loss).

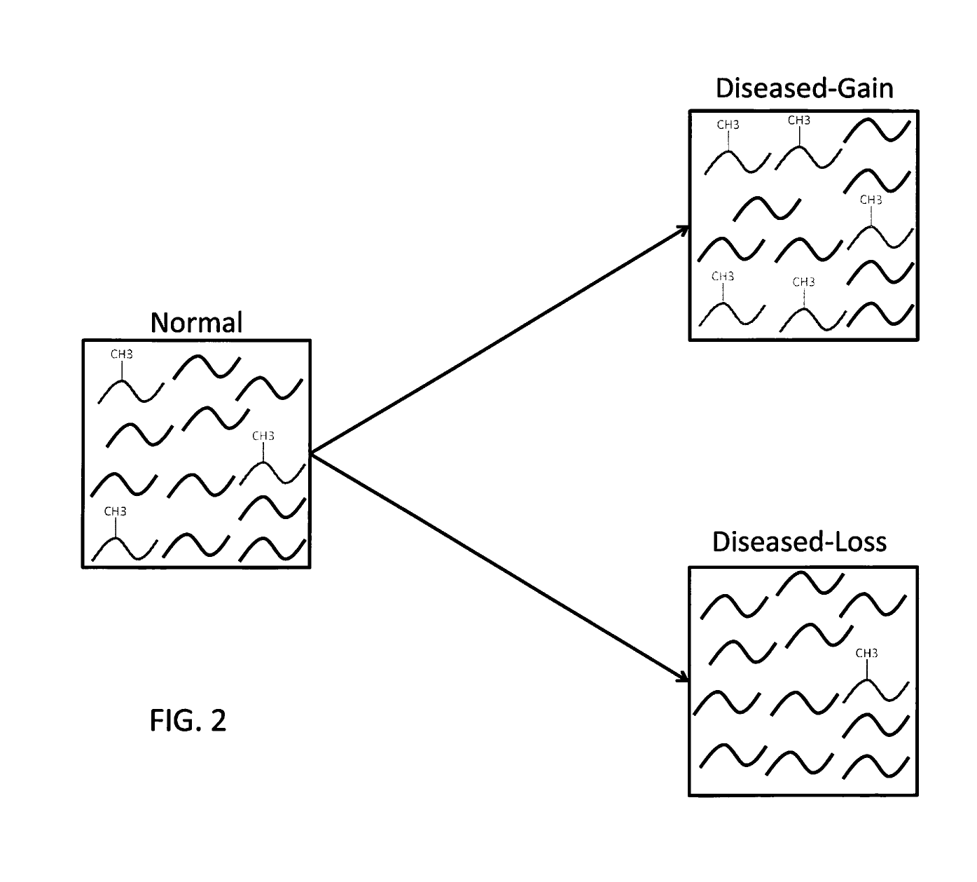

FIG. 3 shows an illustrative embodiment of a system in which certain embodiments of the technology may be implemented.

### DETAILED DESCRIPTION

Technology described herein can be utilized to assess a state of a cell, tissue, body function, medical condition, progression of a medical condition or treatment of a medical condition, for example. Certain embodiments of the technology are useful for (i) determining the likelihood a test subject has a medical disorder or is pre-disposed to having a medical disorder, (ii) determining the presence or absence of a progression of a medical disorder in a test subject, (iii) determining the presence or absence of a response to a therapy administered to a test subject having the medical disorder, (iv) determining whether a dosage of a therapeutic agent administered to a test subject should be increased, decreased or maintained; the like or combination of the foregoing. Various aspects and embodiments of the technology are described hereafter.

### Nucleic Acid

Provided in part herein are methods for nucleic acid quantification. The terms “nucleic acid”, “nucleic acid molecule” and “polynucleotide” may be used interchangeably throughout the disclosure. Non-limiting examples of nucleic acid include deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), DNA or RNA analogs (e.g., containing base analogs, sugar analogs and/or a non-native backbone and the like), RNA/DNA hybrids and polyamide nucleic acids (PNAs). A nucleic acid can be in single-stranded or double-stranded form, and unless otherwise limited, can encompass known analogs of natural nucleotides that can function in a similar manner as naturally occurring nucleotides.

A nucleic acid can be in any form useful for conducting processes herein (e.g., linear, circular, supercoiled, single-stranded, double-stranded and the like). A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell, in certain embodiments. A nucleic acid in some embodiments can be from a single chromosome (e.g., a nucleic acid sample may be from one chromosome of a sample obtained from a diploid organism). The term also may include, as equivalents, derivatives, variants and analogs of RNA or DNA synthesized from nucleotide analogs, single-stranded (e.g., “sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base thymine is replaced with uracil. A nucleic acid may be prepared using a nucleic acid obtained from a subject.

Circulating Cell-Free Nucleic Acid

Nucleic acid can be circulating cell-free nucleic acid in certain embodiments. The terms “circulating cell-free nucleic acid,” “extracellular nucleic acid” and “cell free nucleic acid” as used herein refer to nucleic acid isolated from a source having substantially no cells. Circulating cell-free nucleic acid (ccfNA) can be present in and obtained from blood. Circulating cell-free nucleic acid often includes no detectable cells and may contain cellular elements or cellular remnants. Non-limiting examples of acellular sources for extracellular nucleic acid are blood, blood plasma, blood serum and urine.

Obtaining circulating cell-free nucleic acid includes obtaining a sample directly (e.g., collecting a sample, e.g., a test sample) or obtaining a sample from another who has collected a sample. Without being limited by theory, circulating cell-free nucleic acid may be a product of cell apoptosis and cell breakdown, which provides basis for extracellular nucleic acid often having a series of lengths across a spectrum (e.g., a “ladder”).

Circulating cell-free nucleic acid can include different nucleic acid species, and therefore is referred to herein as “heterogeneous.” For example, blood serum or plasma from a person having cancer can include nucleic acid from cancer cells and nucleic acid from non-cancer cells. In another non-limiting example, blood serum or plasma from a pregnant female can include maternal nucleic acid and fetal nucleic acid. In another non-limiting example, blood serum or plasma from a pregnant female can include maternal nucleic acid, placental nucleic acid and fetal nucleic acid. At least two different nucleic acid species can exist in different amounts in circulating cell-free nucleic acid and sometimes are referred to as minority species and majority species. In certain instances, a minority species of nucleic acid is from an affected cell type (e.g., cancer cell, wasting cell, cell attacked by immune system). In some instances, a minority species of circulating cell-free nucleic acid sometimes is about 1% to about 40% of the overall nucleic acid (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40% of the nucleic acid is minority species nucleic acid). In some embodiments, a minority species of circulating cell-free nucleic acid is of a length of about 500 base pairs or less (e.g., about 80, 85, 90, 91, 92, 93, 94, 95, 96, 97, 98, 99 or 100% of minority species nucleic acid is of a length of about 500 base pairs or less). In some embodiments, a minority species of circulating cell-free nucleic acid is of a length of about 300 base pairs or less (e.g., about 80, 85, 90, 91, 92, 93, 94, 95, 96, 97, 98, 99 or 100% of minority species nucleic acid is of a length of about 300 base pairs or less). In some embodiments, a minority species of circulating cell-free nucleic acid is of a length of about 200 base pairs or less (e.g., about 80, 85, 90, 91, 92, 93, 94, 95, 96, 97, 98, 99 or 100% of minority species nucleic acid is of a length of about 200 base pairs or less). In some embodiments, a minority species of circulating cell-free nucleic acid is of a length of about 150 base pairs or less (e.g., about 80, 85, 90, 91, 92, 93, 94, 95, 96, 97, 98, 99 or 100% of minority species nucleic acid is of a length of about 150 base pairs or less).

Cellular Nucleic Acid

Nucleic acid can be cellular nucleic acid in certain embodiments. The term “cellular nucleic acid” as used herein refers to nucleic acid isolated from a source having intact cells. Non-limiting examples of sources for cellular nucleic acid are blood cells, tissue cells, organ cells, tumor cells, hair cells, skin cells, and bone cells.

In some embodiments, nucleic acid is from peripheral blood mononuclear cells (PBMC). A PBMC is any blood cell having a round nucleus, such as, for example, lymphocytes, monocytes or macrophages. These cells can be extracted from whole blood, for example, using ficoll, a hydrophilic polysaccharide that separates layers of blood, with PBMCs forming a buffy coat under a layer of plasma. Additionally, PBMCs can be extracted from whole blood using a hypotonic lysis which preferentially lyses red blood cells and leaves PBMCs intact, and/or can be extracted using a differential centrifugation process known in the art.

In some embodiments, nucleic acid is from placental cells. The placenta is an organ that connects the developing fetus to the uterine wall to allow nutrient uptake, waste elimination, and gas exchange via the mother's blood supply. The placenta develops from the same sperm and egg cells that form the fetus, and functions as a feto-maternal organ with two components, the fetal part (Chorion frondosum), and the maternal part (Decidua basalis). In some embodiments, nucleic acid is obtained from the fetal part of the placenta. In some embodiments, nucleic acid is obtained from the maternal part of the placenta.

Samples

Nucleic acid in or from a suitable sample can be utilized in a method described herein. A mixture of nucleic acids can comprise two or more nucleic acid fragment species having different nucleotide sequences, different fragment lengths, different origins (e.g., genomic origin, fetal vs. maternal origin, cell or tissue origin, cancer vs. non-cancer origin, tumor vs. non-tumor origin, sample origin, subject origin, and the like), or combinations thereof. In some embodiments, nucleic acid is analyzed in situ (e.g., in a sample; in a subject), in vivo, ex vivo or in vitro.

Nucleic acid often is isolated from a sample obtained from a subject. A subject can be any living or non-living organism, including but not limited to a human, a non-human animal, a plant, a bacterium, a fungus or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be male or female.

Nucleic acid may be isolated from any type of suitable biological specimen or sample (e.g., a test sample). A sample or test sample can be any specimen that is isolated or obtained from a subject (e.g., a human subject, a pregnant female). Non-limiting examples of specimens include fluid or tissue from a subject, including, without limitation, cerebrospinal fluid, spinal fluid, lavage fluid (e.g., bronchoalveolar, gastric, peritoneal, ductal, ear, arthroscopic), urine, feces, sputum, saliva, nasal mucous, prostate fluid, lavage, semen, lymphatic fluid, bile, tears, sweat, breast milk, breast fluid, biopsy sample (e.g., cancer biopsy), cell or tissue sample (e.g., from the liver, lung, spleen, pancreas, colon, skin, bladder, eye, brain, esophagus, head, neck, ovary, testes, prostate, the like or combination thereof). In some embodiments, a biological sample may be blood and sometimes a blood fraction (e.g., plasma or serum). As used herein, the term “blood” encompasses whole blood or any fractions of blood, such as serum and plasma as conventionally defined, for example. Blood or fractions thereof often comprise nucleosomes (e.g., maternal and/or fetal nucleosomes). Nucleosomes comprise nucleic acids and are sometimes cell-free or intracellular. Blood also comprises buffy coats. Buffy coats sometimes are isolated by utilizing a ficoll gradient. Buffy coats can comprise white blood cells (e.g., leukocytes, T-cells, B-cells, platelets, and the like). In some embodiments, buffy coats comprise maternal and/or fetal nucleic acid. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted may be acellular (e.g., cell-free). In some embodiments, a fluid or tissue sample may contain cellular elements or cellular remnants. In some embodiments cancer cells may be included in the sample.

Nucleic Acid Isolation and Processing

Nucleic acid can be isolated using any suitable technique. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 ug/ml Rnase A; a second solution can contain 0.2N NaOH and 1% SDS; and a third solution can contain 3M KOAc, pH 5.5. These procedures can be found in Current Protocols in Molecular Biology, John Wiley & Sons, N.Y., 6.3.1-6.3.6 (1989), incorporated herein in its entirety.

Nucleic acid may be isolated at a different time point as compared to another nucleic acid, where each of the samples is from the same or a different source. A nucleic acid may be from a nucleic acid library, such as a cDNA or RNA library, for example. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid.

The term “amplified” as used herein refers to subjecting a target nucleic acid in a sample to a process that linearly or exponentially generates amplicon nucleic acids having the same or substantially the same nucleotide sequence as the target nucleic acid, or segment thereof. The term “amplified” as used herein can refer to subjecting a target nucleic acid (e.g., in a sample comprising other nucleic acids) to a process that selectively and linearly or exponentially generates amplicon nucleic acids having the same or substantially the same nucleotide sequence as the target nucleic acid, or segment thereof. The term “amplified” as used herein can refer to subjecting a population of nucleic acids to a process that non-selectively and linearly or exponentially generates amplicon nucleic acids having the same or substantially the same nucleotide sequence as nucleic acids, or portions thereof, that were present in the sample prior to amplification. In some embodiments, the term “amplified” refers to a method that comprises a polymerase chain reaction (PCR).

Nucleic acid also may be processed by subjecting nucleic acid to a method that generates nucleic acid fragments, in certain embodiments, before providing nucleic acid for a process described herein. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs. Fragments can be generated by a suitable method known in the art, and the average, mean or nominal length of nucleic acid fragments can be controlled by selecting an appropriate fragment-generating procedure. In certain embodiments, nucleic acid of a relatively shorter length can be utilized to analyze sequences that contain little sequence variation and/or contain relatively large amounts of known nucleotide sequence information. In some embodiments, nucleic acid of a relatively longer length can be utilized to analyze sequences that contain greater sequence variation and/or contain relatively small amounts of nucleotide sequence information.

Nucleic acid fragments may contain overlapping nucleotide sequences, and such overlapping sequences can facilitate construction of a nucleotide sequence of the non-fragmented counterpart nucleic acid, or a segment thereof. For example, one fragment may have subsequences x and y and another fragment may have subsequences y and z, where x, y and z are nucleotide sequences that can be 5 nucleotides in length or greater. Overlap sequence y can be utilized to facilitate construction of the x-y-z nucleotide sequence in nucleic acid from a sample in certain embodiments. Nucleic acid may be partially fragmented (e.g., from an incomplete or terminated specific cleavage reaction) or fully fragmented in certain embodiments.

Nucleic acid can be fragmented by various methods known in the art, which include without limitation, physical, chemical and enzymatic processes. Non-limiting examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected to generate non-specifically cleaved fragments or specifically cleaved fragments. Non-limiting examples of processes that can generate non-specifically cleaved fragment nucleic acid include, without limitation, contacting nucleic acid with apparatus that expose nucleic acid to shearing force (e.g., passing nucleic acid through a syringe needle; use of a French press); exposing nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

As used herein, “fragmentation” or “cleavage” refers to a procedure or conditions in which a nucleic acid molecule, such as a nucleic acid template gene molecule or amplified product thereof, may be severed into two or more smaller nucleic acid molecules. Such fragmentation or cleavage can be sequence specific, base specific, or nonspecific, and can be accomplished by any of a variety of methods, reagents or conditions, including, for example, chemical, enzymatic, physical fragmentation.

As used herein, “fragments”, “cleavage products”, “cleaved products” or grammatical variants thereof, refers to nucleic acid molecules resultant from a fragmentation or cleavage of a nucleic acid template gene molecule or amplified product thereof. While such fragments or cleaved products can refer to all nucleic acid molecules resultant from a cleavage reaction, typically such fragments or cleaved products refer only to nucleic acid molecules resultant from a fragmentation or cleavage of a nucleic acid template gene molecule or the segment of an amplified product thereof containing the corresponding nucleotide sequence of a nucleic acid template gene molecule. For example, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule). Accordingly, fragments can include fragments arising from portions of amplified nucleic acid molecules containing, at least in part, nucleotide sequence information from or based on the representative nucleic acid template molecule.

As used herein, the term “complementary cleavage reactions” refers to cleavage reactions that are carried out on the same nucleic acid using different cleavage reagents or by altering the cleavage specificity of the same cleavage reagent such that alternate cleavage patterns of the same target or reference nucleic acid or protein are generated. In certain embodiments, nucleic acid may be treated with one or more specific cleavage agents (e.g., 1, 2, 3, 4, 5, 6, 7, 8, 9, 10 or more specific cleavage agents) in one or more reaction vessels (e.g., nucleic acid is treated with each specific cleavage agent in a separate vessel).

Nucleic acid may be specifically cleaved or non-specifically cleaved by contacting the nucleic acid with one or more enzymatic cleavage agents (e.g., nucleases, restriction enzymes). The term “specific cleavage agent” as used herein refers to an agent, sometimes a chemical or an enzyme that can cleave a nucleic acid at one or more specific sites. Specific cleavage agents often cleave specifically according to a particular nucleotide sequence at a particular site. Non-specific cleavage agents often cleave nucleic acids at non-specific sites or degrade nucleic acids. Non-specific cleavage agents often degrade nucleic acids by removal of nucleotides from the end (either the 5′ end, 3′ end or both) of a nucleic acid strand. Examples of enzymatic cleavage agents are described herein.

Nucleic acid may be treated with a chemical agent, and the modified nucleic acid may be cleaved. In non-limiting examples, nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any form useful for conducting a sequence analysis or manufacture process described herein, such as solid or liquid form, for example. In certain embodiments, nucleic acid may be provided in a liquid form optionally comprising one or more other components, including without limitation one or more buffers or salts.

### Cell Types

As used herein, a “cell type” refers to a type of cell that can be distinguished from another type of cell. Circulating cell-free nucleic acid can include nucleic acid from several different cell types. Non-limiting examples of cell types that can contribute nucleic acid to circulating cell-free nucleic acid include liver cells (e.g., hepatocytes), lung cells, spleen cells, pancreas cells, colon cells, skin cells, bladder cells, eye cells, brain cells, esophagus cells, cells of the head, cells of the neck, cells of the ovary, cells of the testes, prostate cells, placenta cells, epithelial cells, endothelial cells, adipocyte cells, kidney cells, heart cells, muscle cells, blood cells (e.g., white blood cells), the like and combinations of the foregoing. In some embodiments, cell types that contribute nucleic acid to circulating cell-free nucleic acid analyzed include white blood cells, endothelial cells and hepatocyte liver cells. Different cell types can be screened as part of identifying and selecting nucleic acid loci for which a marker state is the same or substantially the same for a cell type in subjects having a medical condition and for the cell type in subjects not having the medical condition, as described in further detail herein.

A particular cell type sometimes remains the same or substantially the same in subjects having a medical condition and in subjects not having a medical condition. In a non-limiting example, the number of living or viable cells of a particular cell type may be reduced in a cell degenerative condition, and the living, viable cells are not modified, or are not modified significantly, in subjects having the medical condition.

A particular cell type sometimes is modified as part of a medical condition and has one or more different properties than in its original state. In a non-limiting example, a particular cell type may proliferate at a higher than normal rate, may transform into a cell having a different morphology, may transform into a cell that expresses one or more different cell surface markers and/or may become part of a tumor, as part of a cancer condition. In embodiments for which a particular cell type (i.e., a progenitor cell) is modified as part of a medical condition, the marker state for each of the one or more markers assayed often is the same or substantially the same for the particular cell type in subjects having the medical condition and for the particular cell type in subjects not having the medical condition. Thus, the term “cell type” sometimes pertains to a type of cell in subjects not having a medical condition, and to a modified version of the cell in subjects having the medical condition. In some embodiments, a “cell type” is a progenitor cell only and not a modified version arising from the progenitor cell. A “cell type” sometimes pertains to a progenitor cell and a modified cell arising from the progenitor cell. In such embodiments, a marker state for a marker analyzed often is the same or substantially the same for a cell type in subjects having a medical condition and for the cell type in subjects not having the medical condition.

Different cell types can be distinguished by any suitable characteristic, including without limitation, one or more different cell surface markers, one or more different morphological features, one or more different functions, one or more different protein (e.g., histone) modifications and one or more different nucleic acid markers. Non-limiting examples of nucleic acid markers include single-nucleotide polymorphisms (SNPs), methylation state of a nucleic acid locus, short tandem repeats, insertions (e.g., micro-insertions), deletions (micro-deletions) the like and combinations thereof. Non-limiting examples of protein (e.g., histone) modifications include acetylation, methylation, ubiquitylation, phosphorylation, sumoylation, the like and combinations thereof.

As used herein, the term a “related cell type” refers to a cell type having multiple characteristics in common with another cell type. In related cell types, 75% or more cell surface markers sometimes are common to the cell types (e.g., about 80%, 85%, 90% or 95% or more of cell surface markers are common to the related cell types).

### Markers

A marker can be a region of a biological molecule (e.g., nucleic acid, protein or peptide) having, or not having, an epigenetic modification. A marker often is in a particular locus (i.e., region) of a nucleic acid. A nucleic acid locus sometimes is a segment of contiguous nucleotide bases in the nucleic acid, and sometimes the segment is about 5 or more contiguous bases in length (e.g., about 6, 7, 8, 9, 10, 15, 20, 25, 30, 35, 40, 45, 50, 60, 70, 80, 90, 100, 200, 300, 400, 500, 600, 700, 800, 900 or 1,000 or more contiguous bases in length). A nucleic acid locus sometimes includes two or more non-contiguous segments of contiguous bases (e.g., 3, 4, 5, 6, 7, 8, 9, 10 or more non-contiguous stretches of bases). A marker in nucleic acid often includes one or more bases that can be modified by an epigenetic modification, and a marker sometimes is a particular methylation state for a locus.

A marker selected and utilized in a method described herein often is detectable in circulating cell-free nucleic acid and is present in a particular cell type. Without being limited by theory, it is expected that nucleic acid from a particular cell type and bears a particular marker transfers to the blood stream of a subject, and presents in circulating cell-free nucleic acid of the subject. A marker sometimes is specific for a particular cell type or related cell type, and in such embodiments, the marker often is not present at a significant level, is not detectable or is detectable at relatively low levels, in other cell types tested.

A marker state (e.g., methylation state) often is the same or substantially the same for a cell type in subjects having a medical condition and for the cell type in subjects not having the medical condition. A marker state that is substantially the same can include one or more minor modifications. A marker state that is substantially the same as another marker state sometimes includes a small number of differentially methylated nucleotides. In a non-limiting example, about 1, 2 or 3 nucleotides methylated in a locus for one marker are not methylated in a marker that is substantially the same. In another non-limiting example, about 1, 2 or 3 of nucleotides not methylated in a locus for one marker are methylated in a marker that is substantially the same.

The terms “methylation state”, “methylation profile”, or “methylation status,” as used herein to describe the state of methylation of a locus (e.g., a polynucleotide segment(s)), refer to one or more characteristics of a nucleic acid locus relevant to methylation. Non-limiting examples of such characteristics include whether any of the cytosine (C) bases within a locus are methylated, location of methylated C base(s), percentage of methylated C base(s) at a particular locus, and allelic differences in methylation due to, for example, difference in the origin of alleles. The terms above also refer to the relative or absolute amount (e.g., concentration) of methylated C or non-methylated C at a particular locus in a nucleic acid.

As used herein, a “methylated nucleotide” or a “methylated nucleotide base” refers to the presence of a methyl moiety on a nucleotide base, where the methyl moiety is not present in a typical nucleotide base of a newly synthesized nucleic acid. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a nucleotide base incorporated into newly synthesized DNA. Typical nucleoside bases for DNA are thymine, adenine, cytosine and guanine. Typical bases for RNA are uracil, adenine, cytosine and guanine. A “methylation site” is a location in a locus where methylation has occurred, or has the possibility of occurring. A methylation site sometimes is a C base, or each C base, in a locus, and sometimes a methylation site is a CpG site in a locus. Each methylation site in the locus may or may not be methylated. A methylation site can be susceptible to methylation by a naturally occurring event in vivo or by an event that chemically methylates a nucleotide in vitro.

A methylation state sometimes is hypermethylated and sometimes is hypomethylated. For example, if all or a majority of C bases within a locus are methylated, the methylation state can be referred to as “hypermethylated.” In another example, if all or a majority of C bases within a locus are not methylated, the methylation state may be referred to as “hypomethylated.” Likewise, if all or a majority of C bases within a locus are methylated as compared to another polynucleotide from a different region, cell type, tissue or individual, the methylation state is considered hypermethylated compared to the other polynucleotide. Alternatively, if all or a majority of the C bases within a locus are not methylated as compared to another polynucleotide from a different region, cell type, tissue or individual, the methylation state is considered hypomethylated compared to the other polynucleotide, and these polynucleotides are considered “differentially methylated.” Methods and examples of differentially methylated sites in fetal nucleic acid are described in, for example, PCT Publication No. WO2010/033639.

A methylation state of a locus often is hypomethylated or hypermethylated. In some embodiments, there exists a 5′ to 3′ gradient in methylation state across a locus, and sometimes the 5′ end is hypomethylated and the 3′ end is hypermethylated or the 5′ end is hypermethylated and the 3′ end is hypomethylated. In some embodiments, there is no 5′ to 3′ gradient, or 3′ to 5′ gradient, in methylation state.

In some embodiments, a particular methylation state for a locus is defined by, or defined in part by, (i) the number of methylated nucleotides (e.g., methylated C bases), when present, in a locus or loci, (ii) the number of non-methylated nucleotides (e.g., non-methylated C bases), when present, in a locus or loci, or (iii) combination thereof. In certain embodiments, the number of methylated bases and/or the number of non-methylated bases in a locus or loci are factored without regard to the position of the bases that are methylated or non-methylated. In an example, a particular methylation state can be defined by four methylated C bases and six non-methylated C bases in a locus comprising a total of ten C bases, without regard to the position of the bases that are methylated or non-methylated.

A particular methylation state sometimes is defined by, or defined in part by, the position of each methylated nucleotide and/or non-methylated nucleotide in a locus or loci. In some embodiments, a particular methylation state for a locus is defined by (i) the position of methylated nucleotides (e.g., methylated C bases), when present, in a locus or loci, (ii) the position of non-methylated nucleotides (e.g., non-methylated C bases), when present, in a locus or loci, or (iii) combination thereof. In certain embodiments, the position of methylated base and/or the position of each non-methylated base in a locus or loci are factored without regard to the number of the bases that are methylated or non-methylated.

A particular methylation state sometimes is defined by, or defined in part by, (i) the position of each methylated nucleotide and/or non-methylated nucleotide in a locus or loci, and (ii) the number of methylated nucleotides and/or non-methylated nucleotides in a locus or loci. In some embodiments, a particular methylation state for a locus is defined by (i) the position and number of methylated nucleotides (e.g., methylated C bases), when present, in a locus or loci, (ii) the position and number of non-methylated nucleotides (e.g., non-methylated C bases), when present, in a locus or loci, or (iii) combination thereof.

A “nucleic acid comprising one or more CpG sites” or a “CpG-containing sequence” as used herein refers to a segment of DNA sequence at a defined location in the genome (i.e., nucleic acid locus). Typically, a “CpG-containing sequence” is at least 15 nucleotides in length and contains at least one cytosine. Often, a CpG-containing sequence can be at least 30, 50, 80, 100, 150, 200, 250, or 300 nucleotides in length and contains at least 2, 5, 10, 15, 20, 25, or 30 cytosines. For any one “CpG-containing sequence” at a given location (e.g., within a region centering on a given locus), nucleotide sequence variations may exist from individual to individual and from allele to allele even for the same individual. Typically, such a region centering on a defined genetic locus (e.g., a CpG island) contains the locus as well as upstream and/or downstream sequences. Each of the upstream or downstream sequence (counting from the 5′ or 3′ boundary of the genetic locus, respectively) can be as long as 10 kb, in other cases may be as long as 5 kb, 2 kb, 1 kb, 500 bp, 200 bp, or 100 bp. Furthermore, a “CpG-containing sequence” may encompass a nucleotide sequence transcribed or not transcribed for protein production, and the nucleotide sequence can be an inter-gene sequence, intra-gene sequence, protein-coding sequence, a non protein-coding sequence (such as a transcription promoter), or a combination thereof. A “CpG island” as used herein describes a segment of DNA sequence that possesses a functionally or structurally deviated CpG density. A CpG island can be, for example, at least 400 nucleotides in length, have a greater than 50% GC content, and an OCF/ECF ratio greater than 0.6. In some cases a CpG island can be characterized as being at least 200 nucleotides in length, having a greater than 50% GC content, and an OCF/ECF ratio greater than 0.6.

### Marker Detection

Any suitable method or process for detecting a marker, such as a methylation state of a locus, for example, can be utilized. A marker sometimes is detected in cellular nucleic acid (e.g., in situ) and sometime is detected in cellular nucleic acid isolated from cells (e.g., in vitro). A marker sometimes is detected in circulating cell-free nucleic acid (e.g., in situ) and sometimes is detected in circulating cell-free nucleic acid isolated from a subject (e.g., in vitro).

A process for detecting a marker (e.g., methylation state of a locus) sometimes includes amplification of a region of test nucleic acid from a subject. Any suitable amplification process can be utilized, and non-limiting examples of amplification processes include polymerase chain reaction (PCR); ligation amplification (or ligase chain reaction (LCR)); amplification methods based on the use of Q-beta replicase or template-dependent polymerase (see US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, the like and combinations thereof. Methylation sensitive PCR amplification techniques are described herein. Reagents and hardware for conducting nucleic acid amplification are commercially available.

A process for detecting a methylation state of a locus for a marker sometimes includes treatment of a nucleic acid with a suitable agent or agents that differentially modify the nucleic acid according to whether nucleotides are methylated nucleotides or non-methylated nucleotides. A nucleic acid treated by such an agent includes without limitation sample nucleic acid, amplified nucleic acid, nucleic acid treated with an agent that selective cleaves methylated nucleotides or non-methylated nucleotides (described herein), the like and combinations of the foregoing. An agent that selective modifies nucleotides based on methylation state sometimes converts a methylated cytosine nucleotide to uracil nucleotide. Methods for modifying a nucleic acid molecule in a manner that reflects the methylation pattern of the nucleic acid molecule are known in the art, and non-limiting examples are described in U.S. Pat. No. 5,786,146 and U.S. patent publications 20030180779 and 20030082600. For example, non-methylated cytosine nucleotides in a nucleic acid can be converted to uracil by bisulfite treatment, which does not modify methylated cytosine.

A cleavage agent may be utilized, as part of a process for detecting a methylation state of a locus for a marker, that specifically and differentially cleaves according to non-modified nucleotides and modified nucleotides in a nucleic acid, where the modified nucleotides are modification products of methylated nucleotides. A nucleic acid treated by such an agent includes, without limitation, sample nucleic acid treated with an agent that selectively modifies methylated nucleotides or non-methylated nucleotides (e.g., the cleavage agent cleaves specifically according to unmodified nucleotides and modified nucleotides) and amplification products thereof. In some embodiments, a cleavage agent specifically cleaves at or near a cleavage site comprising one or more uracil nucleotides that have been converted from methylated cytosine. Nucleic acid sometimes is exposed to one or more of such cleavage agents prior to amplification, and sometimes nucleic acid is exposed to one or more of such cleavage agents following amplification. In some embodiments, nucleic acid is exposed to one or more of such cleavage agents prior to amplification and following amplification. Selection and use of such cleavage agents are known, and non-limiting examples include certain restriction enzymes.

Non-limiting examples of restriction enzymes include DNase (e.g., DNase I, II); RNase (e.g., RNase E, F, H, P); CLEAVASE enzyme; TAQ DNA polymerase; E. coli DNA polymerase I and eukaryotic structure-specific endonucleases; murine FEN-1 endonucleases; type I, II or III restriction endonucleases (i.e. restriction enzymes) such as Acc I, Acil, Afl III, Alu I, Alw44 I, Apa I, Asn I, Ava I, Ava II, BamH I, Ban II, Bcl I, Bgl I. Bgl II, Bln I, Bsm I, BssH II, BstE II, BstUI, Cfo I, Cla I, Dde I, Dpn I, Dra I, EclX I, EcoR I, EcoR I, EcoR II, EcoR V, Hae II, Hae II, Hhal, Hind III, Hind III, Hpa I, HinP1I, Hpa II, Kpn I, Ksp I, MaeII, McrBC, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I; glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease I, exonuclease II, exonuclease III, exonuclease IV, exonuclease V, exonuclease VI, exonuclease VII, exonuclease VIII); ribozymes, and DNAzymes. One or more restriction enzymes sometimes are utilized under conditions that permit cleavage of target nucleic acid with about 90% to about 100% efficiency or about 98% to about 100% efficiency (e.g., about 95%, 96%, 97%, 98%, 99% efficiency).

A process for detecting a methylation state of a locus for a marker sometimes includes treatment of nucleic acid with an agent that specifically and differentially cleaves according to methylation state at a particular locus. A nucleic acid treated by such an agent includes, without limitation, sample nucleic acid and amplified sample nucleic acid. Nucleic acid can be exposed to one or more of such cleavage agents prior to amplification. Selection and use of methylation-sensitive cleavage agents are known, and non-limiting examples of such agents are methylation-sensitive restriction enzymes.

Certain methylation-sensitive restriction enzymes preferentially and/or substantially cleave (e.g., digest) at a non-methylated recognition sequence, and some methylation-sensitive restriction enzymes preferentially and/or substantially cleave at a methylated recognition sequence. Non-limiting examples of enzymes that digest nucleic acid according to a methylated recognition sequence include DpnI, which cuts at a recognition sequence GATC, and McrBC, which cuts DNA containing modified cytosines (New England BioLabs®, Inc, Beverly, Mass.). Non-limiting examples of enzymes that digest nucleic acid according to a non-methylated recognition sequence include HpaII, HinP1I, Hhal, MaeII, BstUI and Acil. In some embodiments, combinations of two or more methylation-sensitive enzymes that digest non-methylated DNA are used. In some embodiments, HpaII, which cleaves the non-methylated sequence CCGG, is used. In some embodiments, Hhal, which cleaves the non-methylated sequence GCGC, is used. Both enzymes are available from New England BioLabs®, Inc (Beverly, Mass.). One or more methylation-specific enzymes sometimes are utilized under conditions that permit cleavage of target nucleic acid with about 90% to about 100% efficiency or about 98% to about 100% efficiency (e.g., about 95%, 96%, 97%, 98%, 99% efficiency).

A process for detecting a marker sometimes includes incorporation of a detectable label into nucleic acid (e.g., sample nucleic acid or modified version thereof or amplification product of the foregoing). Non-limiting examples of detectable labels include fluorescent labels such as organic fluorophores, lanthanide fluorophores (chelated lanthanides; dipicolinate-based Terbium (III) chelators), transition metal-ligand complex fluorophores (e.g., complexes of Ruthenium, Rhenium or Osmium); quantum dot fluorophores, isothiocyanate fluorophore derivatives (e.g., FITC, TRITC), succinimidyl ester fluorophores (e.g., NHS-fluorescein), maleimide-activated fluorophores (e.g., fluorescein-5-maleimide), and amidite fluorophores (e.g., 6-FAM phosphoramidite); radioactive isotopes (e.g., I-125, I-131, S-35, P-31, P-32, C-14, H-3, Be-7, Mg-28, Co-57, Zn-65, Cu-67, Ge-68, Sr-82, Rb-83, Tc-95m, Tc-96, Pd-103, Cd-109, and Xe-127); light scattering labels (e.g., light scattering gold nanorods, resonance light scattering particles); an enzymic or protein label (e.g., green fluorescence protein (GFP), peroxidase); or other chromogenic label or dye (e.g., cyanine). Non-limiting examples of organic fluorophores include xanthene derivatives (e.g., fluorescein, rhodamine, Oregon green, eosin, Texas red); cyanine derivatives (e.g., cyanine, indocarbocyanine, oxacarbocyanine, thiacarbocyanine, merocyanine); naphthalene derivatives (dansyl, prodan derivatives); coumarin derivatives; oxadiazole derivatives (e.g., pyridyloxazole, nitrobenzoxadiazole, benzoxadiazole); pyrene derivatives (e.g., cascade blue); oxazine derivatives (e.g., Nile red, Nile blue, cresyl violet, oxazine 170); acridine derivatives (e.g., proflavin, acridine orange, acridine yellow); arylmethine derivatives (e.g., auramine, crystal violet, malachite green); and tetrapyrrole derivatives (e.g., porphin, phtalocyanine, bilirubin). A detectable label sometimes is a particular polynucleotide tag, which can be detected in a suitable manner. In some embodiments, a polynucleotide detectable label is cleaved, amplified or hybridized to a labeled probe, for example, and then detected.

A process for detecting a marker sometimes includes an amplification process, use of an agent that differentially modifies according to the methylation state of one or more nucleotides, a specific cleavage agent, incorporation of a detectable label, the like or a combination of the foregoing. For embodiments that include more than one of such processes, the processes may be implemented in any suitable order. In certain embodiments, (i) sample nucleic acid (e.g., cellular nucleic acid or circulating cell-free nucleic acid) is digested with a cleavage agent that specifically cleaves according to whether a nucleotide is methylated or not methylated, and (ii) cleaved and/or non-cleaved nucleic acid is amplified, and amplicons are detected and quantified. In some embodiments, (i) sample nucleic acid (e.g., cellular nucleic acid or circulating cell-free nucleic acid) is modified with an agent that specifically modifies according to methylation state (e.g., bisulfite), (ii) the modified nucleic acid is cleaved by a cleavage agent that specifically cleaves according to whether a nucleotide is modified or unmodified, and (iii) cleaved and/or non-cleaved nucleic acid is amplified, and amplicons are detected and quantified.

Specific technologies for detecting markers are known in the art. Non-limiting examples of such technologies are described herein (e.g., immunoprecipitation and others).

### Marker Selection

Markers can be selected according to whether they meet one or more criteria described herein. In certain embodiments, provided is a method for preparing a collection of nucleic acid markers, comprising: (a) determining the methylation state of multiple loci in nucleic acid from multiple cell types (e.g., in cellular nucleic acid from particular cell types) from multiple subjects; and (b) selecting loci for which the methylation state is the same or substantially the same for a cell type in subjects having a medical condition and for the cell type in subjects not having the medical condition; whereby a collection of nucleic acid markers is prepared. Such a method sometimes includes synthesizing one or more loci in the collection of markers using a suitable nucleic acid synthesis process. A process can be selected that yields loci that include no methyl moieties or loci that include one or more methyl moieties present in the marker. In some embodiments, synthesizing the one or more loci includes amplifying a portion of nucleic acid from a subject comprising one of the loci using a suitable nucleic acid amplification process.

Also provided is a method for obtaining a collection of amplification primers, comprising: (a) determining the methylation state of multiple loci in nucleic acid from multiple cell types (e.g., in cellular nucleic acid from particular cell types) from multiple subjects; (b) selecting loci for which the methylation state is the same or substantially the same for a cell type in subjects having a medical condition and for the cell type in subjects not having the medical condition; and (c) designing amplification primers, each of which primers is capable of amplifying each of the loci selected in (b); whereby a collection of amplification primers is obtained. Such a method sometimes includes synthesizing the collection of amplification primers using a suitable nucleic acid synthesis process.

Loci for which the methylation state is the same or substantially the same for a cell type in subjects having a medical condition (“population I”) and for the cell type in subjects not having the medical condition (“population II”) can be identified and selected using a suitable process. In some embodiments, a locus is selected where the methylation state of the locus is substantially the same in the cell type in about 80% or more of population I and about 80% or more of population II. The foregoing threshold of about 80% or more can be the same or different for population I and population II, and sometimes is about 85%, 90%, 95%, 96%, 97%, 98% or 99% or more of population I and/or population II.

Loci for which the methylation state is (i) the same or substantially the same for a cell type in population I and population II, as addressed above, and (ii) not the same in other cell types, can be identified and selected using a suitable process. In the latter embodiments, a marker meeting such criteria can be viewed as a cell specific marker. In some embodiments, the methylation state of a locus in a specific cell type is not the same in other cell types within a tissue comprising that specific cell type.

In some embodiments, the methylation state of a locus is not the same in other cell types tested or analyzed. In some embodiments, the methylation state of a locus is not the same in other tissues tested. In certain embodiments, the methylation state of a locus is not the same in other cell types tested or analyzed, and the methylation state of the locus is not the same in other tissues tested. In the latter embodiments, a marker meeting such criteria can be viewed as a cell specific and tissue specific marker. A non-limiting group of markers is provided in Example 1 herein.

### Medical Disorders and Medical Conditions

Methods described herein can be applicable to any suitable medical disorder or medical condition. Non-limiting examples of medical disorders and medical conditions include cell proliferative disorders and conditions, wasting disorders and conditions, degenerative disorders and conditions, autoimmune disorders and conditions, pre-eclampsia, chemical or environmental toxicity, liver damage or disease, kidney damage or disease, vascular disease, high blood pressure, and myocardial infarction.

In some embodiments, a cell proliferative disorder or condition is a cancer of the liver, lung, spleen, pancreas, colon, skin, bladder, eye, brain, esophagus, head, neck, ovary, testes, prostate, the like or combination thereof. Non-limiting examples of cancers include hematopoietic neoplastic disorders, which are diseases involving hyperplastic/neoplastic cells of hematopoietic origin (e.g., arising from myeloid, lymphoid or erythroid lineages, or precursor cells thereof), and can arise from poorly differentiated acute leukemias (e.g., erythroblastic leukemia and acute megakaryoblastic leukemia). Certain myeloid disorders include, but are not limited to, acute promyeloid leukemia (APML), acute myelogenous leukemia (AML) and chronic myelogenous leukemia (CML). Certain lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Certain forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. A cell proliferative disorder sometimes is a non-endocrine tumor or endocrine tumor. Illustrative examples of non-endocrine tumors include, but are not limited to, adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor sometimes is an islet cell tumor.

In some embodiments, a wasting disorder or condition, or degenerative disorder or condition, is cirrhosis, amyotrophic lateral sclerosis (ALS), Alzheimer's disease, Parkinson's disease, multiple system atrophy, atherosclerosis, progressive supranuclear palsy, Tay-Sachs disease, diabetes, heart disease, keratoconus, inflammatory bowel disease (IBD), prostatitis, osteoarthritis, osteoporosis, rheumatoid arthritis, Huntington's disease, chronic traumatic encephalopathy, chronic obstructive pulmonary disease (COPD), tuberculosis, chronic diarrhea, acquired immune deficiency syndrome (AIDS), superior mesenteric artery syndrome, the like or combination thereof.

In some embodiments, an autoimmune disorder or condition is acute disseminated encephalomyelitis (ADEM), Addison's disease, alopecia greata, ankylosing spondylitis, antiphospholipid antibody syndrome (APS), autoimmune hemolytic anemia, autoimmune hepatitis, autoimmune inner ear disease, bullous pemphigoid, coeliac disease, Chagas disease, chronic obstructive pulmonary disease, Crohns Disease (a type of idiopathic inflammatory bowel disease “IBD”), dermatomyositis, diabetes mellitus type 1, endometriosis, Goodpasture's syndrome, Graves' disease, Guillain-Barrè syndrome (GBS), Hashimoto's disease, hidradenitis suppurativa, idiopathic thrombocytopenic purpura, interstitial cystitis, Lupus erythematosus, mixed connective tissue disease, morphea, multiple sclerosis (MS), myasthenia gravis, narcolepsy, euromyotonia, pemphigus vulgaris, pernicious anaemia, polymyositis, primary biliary cirrhosis, rheumatoid arthritis, schizophrenia, scleroderma, Sjögren's syndrome, temporal arteritis (also known as “giant cell arteritis”), ulcerative colitis (a type of idiopathic inflammatory bowel disease “IBD”), vasculitis, vitiligo, Wegener's granulomatosis, the like or combination thereof.

### Marker Quantification

A marker can be quantified using a suitable quantification process that yields an amount of the marker. A detection technology used to detect a particular marker sometimes is used to quantify an amount of the marker. An amount of a marker sometimes is a raw value or experimental value, and sometimes is a processed value, non-limiting examples of the latter including a scaled amount, nominal amount, maximum amount, minimum amount, normalized amount or average amount (e.g., mean, median, mode, range midpoint). A processed amount quantified for one population (e.g., population I) often is processed in the same manner as a processed amount quantified for another population (e.g., population II). For example, a mean of the marker amounts determined for population I can be compared to a mean of the marker amounts determined for population II. An amount of a marker can be expressed as an absolute amount, non-limiting examples of which include weight (e.g., grams or fraction thereof (e.g., micrograms, nanograms, femptograms)) and number of copies. An amount of a marker sometimes is expressed as a relative amount, non-limiting examples of which include a fractional amount (e.g., percentage), a ratio and a concentration, and sometimes a normalized amount or average amount (e.g., median, mean, mode, range midpoint). A relative amount sometimes is expressed as a fraction or ratio of one marker to total nucleic acid or total amount of markers for a locus. For example, where 600 copies of a hypomethylated methylation state for a locus (hypomethylated marker) and 300 copies of a hypermethylated methylation state for the same locus (hypermethylated marker) are quantified, the amount of hypomethylated marker relative to total marker is two-thirds. A relative amount sometimes is expressed as a fraction or ratio of one marker to another marker (e.g., another marker for the locus). For example, where 600 copies of a hypomethylated methylation state for a locus (hypomethylated marker) and 300 copies of a hypermethylated methylation state for the same locus (hypermethylated marker) are quantified, the amount of hypermethylated marker relative to hypomethylated marker is one-half. In some embodiments, a relative amount is expressed relative to the highest detection limit or lowest detection limit of an assay used to quantify a marker.

In some embodiments, the amount of a particular marker is relatively low in circulatory cell-free nucleic acid (“ccfNA”) in subjects not having a medical condition (“population II”). In embodiments where an amount of marker is expressed as a fraction or ratio relative to the amount of a reference (e.g., total nucleic acid, to total marker for a locus, or to another marker or set of markers), the amount of a particular marker in ccfNA sometimes is about 20%, or less, of the amount of the reference. For example, the amount of a particular marker in ccfNA sometimes is about 15%, 10%, 5%, 4%, 3%, 2%, 1%, or less, of the amount of the reference. In some embodiments, the amount of a particular marker in ccfNA is 5-fold lower, or less, than the amount of the reference. For example, the amount of a particular marker in ccfNA sometimes is about 10-fold lower, 20-fold lower, 50-fold lower, 75-fold lower, 100-fold lower, 500-fold lower, 1,000-fold lower, 5,000-fold lower, 10,000-fold lower, 50,000-fold lower, 100,000-fold lower, 500,000-fold lower or 1 million-fold lower, or less, than the amount of the reference.

In some embodiments, the amount of a marker quantified in ccfNA in subjects having a medical condition (“population I”) is significantly greater than the amount of the marker quantified in ccfNA in subjects not having the medical condition (“population II”). In some embodiments, an amount of a marker in ccfNA in population II (e.g., absolute amount, relative amount) is about 20%, or less, of the amount of the marker in ccfNA in population I (e.g., the amount in population II is about 15%, 10%, 5%, 4%, 3%, 2%, 1%, or less, of the amount in population I). An amount of a marker in ccfNA in population II sometimes is about 5-fold lower, or less, than the amount of the marker in ccfNA in population I (e.g., about 10-fold lower, 20-folder lower, 50-fold lower, 75-fold lower, 100-fold or lower, 500-fold lower, 1,000-fold lower, 5,000-fold lower, 10,000-fold lower, 50,000-fold lower, 100,000-fold lower, 500,000-fold lower, 1 million-fold lower or less).

An amount of a particular marker sometimes is below a detectable limit of a particular detection assay in ccfNA of population II, and is detectable in ccfNA in population I. Such a marker can be utilized in an assay for which quantification of the marker is digital (i.e., detected or not detected).

An amount of a particular marker sometimes is detected at an assay signal in ccfNA of subjects in population II that is significantly less than the maximum assay signal of the assay, or significantly less than the assay signal at which the marker is detected in ccfNA for subjects in population I. In some embodiments, a marker is detected in ccfNA of population II at or near a lower detection limit of an assay. In some embodiments, the amount of a marker in ccfNA in population II is detected with a signal that is about 20%, or less, of the maximum assay signal or of the assay signal at which the marker is detected in ccfNA for subjects in population I (e.g., the amount in population II is detected with a signal at about 15%, 10%, 5%, 4%, 3%, 2%, 1%, or less, of the assay signal). An amount of a marker in ccfNA in population II sometimes is detected with a signal that is about 5-fold lower, or less, than the maximum assay signal or of the assay signal at which the marker is detected in ccfNA for subjects in population I (e.g., about 10-fold lower, 20-folder lower, 50-fold lower, 75-fold lower, 100-fold or lower, 500-fold lower, 1,000-fold lower, 5,000-fold lower, 10,000-fold lower, 50,000-fold lower, 100,000-fold lower, 500,000-fold lower, 1 million-fold lower, or less, than the assay signal).

In some embodiments, the amount of a particular marker is relatively high in ccfNA in subjects not having a medical condition (“population II”). In embodiments where an amount of marker is expressed as a fraction or ratio relative to an amount of a reference (e.g., total nucleic acid, to total marker for a locus, or to another marker or set of markers), the amount of a particular marker in ccfNA sometimes is about 80%, or more, of the amount of the reference. For example, the amount of a particular marker in ccfNA sometimes is about 85%, 90%, 95%, 96%, 97%, 98%, 99%, or more, of the amount of the reference.

In some embodiments, the amount of a marker quantified in ccfNA in subjects having a medical condition (“population I”) is significantly less than the amount of the marker quantified in ccfNA in subjects not having the medical condition (“population II”). In some embodiments, an amount of a marker in ccfNA in population I (e.g., absolute amount, relative amount) is about 20%, or less, of the amount of the marker in ccfNA in population II (e.g., the amount in population I is about 15%, 10%, 5%, 4%, 3%, 2%, 1%, or less, of the amount in population II). An amount of a marker in ccfNA in population I sometimes is about 5-fold lower, or less, than the amount of the marker in ccfNA in population II (e.g., about 10-fold lower, 20-folder lower, 50-fold lower, 75-fold lower, 100-fold or lower, 500-fold lower, 1,000-fold lower, 5,000-fold lower, 10,000-fold lower, 50,000-fold lower, 100,000-fold lower, 500,000-fold lower, 1 million-fold lower or less).

An amount of a particular marker sometimes is below a detectable limit of a particular detection assay in ccfNA of population I, and is detectable in ccfNA in population II. Such a marker can be utilized in an assay for which quantification of the marker is digital (i.e., detected or not detected).

An amount of a particular marker sometimes is detected at an assay signal in ccfNA of subjects in population I that is significantly less than the maximum assay signal of the assay, or significantly less than the assay signal at which the marker is detected in ccfNA for subjects in population II. In some embodiments, a marker is detected in ccfNA of population I at or near a lower detection limit of an assay. In some embodiments, the amount of a marker in ccfNA in population I is detected with a signal that is about 20%, or less, of the maximum assay signal or of the assay signal at which the marker is detected in ccfNA for subjects in population II (e.g., the amount in population I is detected with a signal at about 15%, 10%, 5%, 4%, 3%, 2%, 1%, or less, of the assay signal). An amount of a marker in ccfNA in population I sometimes is detected with a signal that is about 5-fold lower, or less, than the maximum assay signal or of the assay signal at which the marker is detected in ccfNA for subjects in population II (e.g., about 10-fold lower, 20-folder lower, 50-fold lower, 75-fold lower, 100-fold or lower, 500-fold lower, 1,000-fold lower, 5,000-fold lower, 10,000-fold lower, 50,000-fold lower, 100,000-fold lower, 500,000-fold lower, 1 million-fold lower, or less, than the assay signal).

As part of an amplification process, template nucleic acid (e.g., an aliquot of template nucleic acid) can be amplified in the presence of a competitor, which can facilitate quantification of the template. A competitor often is an alternate template that shares structural features of the template (e.g., the template and competitor may differ by a small number of nucleotides or a single nucleotide), and a competitor can provide for quantification of the number of template copies. Design and use of competitor oligonucleotides is known, and described, for example, in International Application Publication No. WO 2012/149339 published on Nov. 1, 2012 (International Application No. PCT/US2012/035479 filed on Apr. 27, 2012).

Specific technologies for quantifying markers are known in the art. Non-limiting examples of such technologies are described herein.

### Use of Marker Quantification for Making a Determination

A marker or set of markers may be quantified as described herein for the purpose of making a determination. Non-limiting examples of a determination include (i) determining the likelihood a test subject has a medical disorder or is pre-disposed to having a medical disorder, (ii) determining the presence or absence of a progression of a medical disorder in a test subject, (iii) determining the presence or absence of a response to a therapy administered to a test subject having the medical disorder, (iv) determining whether a dosage of a therapeutic agent administered to a test subject should be increased, decreased or maintained; the like or combination of the foregoing.

For embodiments in which multiple markers are quantified, the amount of one marker, the amounts of a subset of the markers, or the amounts of all of the markers assayed, can be utilized for rendering a determination. For embodiments in which amounts of multiple markers are utilized for rendering a determination, one, some or all of the amounts may be processed (e.g., scaled amount, nominal amount, maximum amount, minimum amount, normalized amount or average amount (e.g., mean, median, mode, range midpoint)). For embodiments in which the amounts of multiple markers are utilized for rendering a determination, the amounts may be utilized with equal weighting or the amounts for different markers may be assigned one or more different weightings and then utilized for rendering a determination (e.g., one or more subsets of markers may be assigned, independently, different weightings).

As explained herein, the presence or absence of a change in a marker state (e.g., a methylation state of a locus) typically is not determined as part of marker quantification. Thus, one or more markers often are quantified by determining the amount of each of the one or more markers in ccfNA without determining the presence of absence of a change in the state of each of the one or more markers. One or more markers often are quantified by determining the amount of markers having the same or substantially the same methylation state for a cell type in subjects having a medical condition and for the cell type in subjects not having the medical condition, and not by analyzing markers having a different methylation state in a cell type for subjects having a medical condition and subjects not having the medical condition. A determination based on a marker quantification often is made without determining the presence or absence of a change in a marker state or by analyzing markers having a different methylation state at a particular locus in different populations. An entire process that includes quantifying a marker sometimes is conducted without determining the presence or absence of a change in a marker state or analyzing markers having a different methylation state at a particular locus in different populations.

Markers analyzed as part of a method herein sometimes are not in a chromosome or chromosome segment (i) amplified or deleted in a cell type in subjects having a medical condition (e.g., disease population; cancer population), and (ii) not amplified or not deleted in the cell type in subjects not having the medical condition (e.g., healthy population). The amount of certain markers analyzed as a part of a method herein may be increased or decreased in ccfNA, and such markers sometimes are not in a chromosome or chromosome segment that is amplified or deleted in certain subjects, where the chromosome or chromosome segment amplification or deletion is associated with a medical condition (e.g., cancer). Markers analyzed as part of a method herein sometimes are in a chromosome or chromosome segment having the same or substantially the same dosage in a disease population (e.g., cancer population) and a healthy population. In some embodiments, markers analyzed as part of a method herein are in a chromosome or chromosome segment having the same or substantially the same dosage in a cell type in a disease population (e.g., cancer population) and the same cell type in a healthy population.

The number of markers for which quantifications are provided can be chosen to permit a determination described herein with a confidence level of about 90% or greater. In some embodiments, the number of markers quantified permit a determination with a confidence level of about 91%, 92%, 93%, 94%, 95%, 96%, 97%, 98%, 99% or greater than 99%.

A process for rendering a determination sometimes includes comparing a quantification of one or more markers to a predetermined value (e.g., cutoff value), such as a predetermined value present in a table or chart (e.g., lookup table or lookup chart). For embodiments in which multiple markers are quantified, a table or chart may include a predetermined value for some or all of the markers. In some embodiments, amounts for the collection of markers quantified can be processed and a composite or relative amount can be rendered and compared to a composite or relative amount in a chart or table. Non-limiting examples of a composite or relative amount include a scaled amount, nominal amount, maximum amount, minimum amount, normalized amount or average amount (e.g., mean, median, mode, range midpoint)). A determination often is rendered based on whether the amount(s) of the marker(s), or processed version thereof, is greater than, greater than or equal to, less than, or less than or equal to, the predetermined amount(s). Thus, in some embodiments, (i) determining the likelihood a test subject has a medical disorder or is pre-disposed to having a medical disorder, (ii) determining the presence or absence of a progression of a medical disorder in a test subject, (iii) determining the presence or absence of a response to a therapy administered to a test subject having the medical disorder, or (iv) determining whether a dosage of a therapeutic agent administered to a test subject should be increased, decreased or maintained, includes comparing a quantified amount of a marker, or processed version thereof, to a predetermined amount in a table or chart.

A process for rendering a determination sometimes includes comparing a quantification of one or more markers using samples obtained at different time points. A determination can be rendered based on a ratio or fraction of the amount(s) quantified for the marker(s) calculated for two or more time points. A determination can be rendered based on a profile, graph, plot or rate of change of the amount(s) quantified for the marker(s) calculated for different time points. A ratio, fraction, profile or rate of change can be calculated for marker quantifications at different time points using methods known in the art, and such values sometimes are compared to a predetermined value (e.g., cutoff value) in a lookup table or chart. A determination sometimes is rendered based on whether the ratio, fraction, profile or rate of change calculated is greater than or less than the predetermined value.

A determination sometimes is utilized as part of a diagnosis. For example, a health care provider may analyze a determination and provide a diagnosis based on, or based in part on, the determination. A determination is not a diagnosis, or is not used for rendering a diagnosis, in some embodiments (e.g., a determination provides an indication of a state of a subject).

A quantification or a determination sometimes comprises a call or score. A quantification sometimes is a genotype and a determination sometimes is a phenotype, In some embodiments, a quantification or determination is provided with an associated level of accuracy, precision and or confidence. A level of accuracy, precision and/or confidence sometimes is a call rate (e.g., about 90% to about 100% correct call rate), a coefficient of variance (CV), an uncertainty value, a confidence level (e.g., a confidence level of about 95% to about 99%)), the like or combination thereof.

A determination sometimes is expressed as a risk or probability (e.g., of the presence or absence of a medical disorder). A quantification or determination sometimes comprises one or more numerical values generated using a method described herein in the context of one or more considerations of probability. A consideration of risk or probability can include, but is not limited to: an uncertainty value, a measure of variability, confidence level, sensitivity, specificity, standard deviation, coefficient of variation (CV) and/or confidence level, Z-scores, Chi values, Phi values, the like or combinations thereof. A consideration of probability can facilitate determining whether a subject is at risk of having, or has, a medical disorder, for example.

A determination sometimes includes a null result. A null result sometimes is a data point between two clusters, or sometimes is a numerical value with a standard deviation that encompasses values for both the presence and absence of an outcome. In some embodiments, a determination indicative of a null result still is useful, and the null result can indicate the need for additional information, a repeat of data generation and/or analysis for rendering a determination.

A determination can be expressed in any suitable form, and sometimes is expressed as a probability (e.g., odds ratio, p-value), likelihood, value in or out of a cluster, value over or under a threshold value, value within a range (e.g., a threshold range), value with a measure of variance or confidence, or risk factor, associated with the presence or absence of a genetic variation for a subject or sample. In certain embodiments, comparison between samples allows confirmation of sample identity (e.g., allows identification of repeated samples and/or samples that have been mixed up (e.g., mislabeled, combined, and the like)).

In some embodiments, a determination comprises a value above or below a predetermined threshold or cutoff value (e.g., greater than 1, less than 1), and an uncertainty or confidence level associated with the value. A determination also can describe an assumption used in data processing. In certain embodiments, a determination comprises a value that falls within or outside a predetermined range of values (e.g., a threshold range) and the associated uncertainty or confidence level for that value being inside or outside the range. In some embodiments, a determination comprises a value that is equal to a predetermined value (e.g., equal to 1, equal to zero), or is equal to a value within a predetermined value range, and its associated uncertainty or confidence level for that value being equal or within or outside a range. A determination sometimes is graphically represented as a plot (e.g., profile plot).

Different methods for generating a determination sometimes can produce different types of results. A determination can lead to four types of scores or calls: true positive, false positive, true negative and false negative. Thus, a determination can be characterized as a true positive, true negative, false positive or false negative in some embodiments. The term “true positive” as used herein refers to a correctly rendered positive determination for a subject. The term “false positive” as used herein refers to an incorrectly rendered positive determination for a subject. The term “true negative” as used herein refers to a correctly rendered negative determination for a subject. The term “false negative” as used herein refers to an incorrectly rendered negative determination for a subject. Two measures of performance for any given method can be calculated based on ratios of these occurrences: (i) a sensitivity value, which generally is the fraction of predicted positives that are correctly identified as being positives; and (ii) a specificity value, which generally is the fraction of predicted negatives correctly identified as being negative.

The term “sensitivity” as used herein refers to the number of true positives divided by the number of true positives plus the number of false negatives, where sensitivity (sens) may be within the range of 0≦sens≦1. Ideally, the number of false negatives equal zero or close to zero, such that an incorrect negative determination is not provided or minimized. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where specificity (spec) may be within the range of 0≦spec≦1. Ideally, the number of false positives equal zero or close to zero, such that an incorrect positive determination is not provided or is minimized.

In certain embodiments, one or more of sensitivity, specificity and/or confidence level are expressed as a percentage. In some embodiments, the percentage, independently for each variable, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome is not due to chance) in certain embodiments is expressed as a Z-score, a p-value, or the results of a t-test. In some embodiments, a measured variance, confidence interval, sensitivity, specificity and the like (e.g., referred to collectively as confidence parameters) is generated for a determination.

A method (e.g., a method using a particular set of markers) that has sensitivity and specificity equaling one, or 100%, or near one (e.g., between about 90% to about 99%) sometimes is selected for rendering a determination. In some embodiments, a method having a sensitivity equaling 1, or 100% is selected, and in certain embodiments, a method having a sensitivity near 1 is selected (e.g., a sensitivity of about 90%, a sensitivity of about 91%, a sensitivity of about 92%, a sensitivity of about 93%, a sensitivity of about 94%, a sensitivity of about 95%, a sensitivity of about 96%, a sensitivity of about 97%, a sensitivity of about 98%, or a sensitivity of about 99%). In some embodiments, a method having a specificity equaling 1, or 100% is selected, and in certain embodiments, a method having a specificity near 1 is selected (e.g., a specificity of about 90%, a specificity of about 91%, a specificity of about 92%, a specificity of about 93%, a specificity of about 94%, a specificity of about 95%, a specificity of about 96%, a specificity of about 97%, a specificity of about 98%, or a specificity of about 99%).

A process described herein for rendering a quantification and/or a determination can be transformative. For example, a marker from a particular cell type in ccfNA can be transformed by a method provided herein into a representation of the amount of nucleic acid from the cell type being dosed into the bloodstream of a subject. Such a transformed representation often is specifically utilized as part of making a determination described herein.

### Medical Disorders and Medical Conditions

Methods described herein can be applicable to any suitable medical disorder or medical condition. Non-limiting examples of medical disorders and medical conditions include cell proliferative disorders and conditions, wasting disorders and conditions, degenerative disorders and conditions, autoimmune disorders and conditions, pre-eclampsia, chemical or environmental toxicity, liver damage or disease, kidney damage or disease, vascular disease, high blood pressure, and myocardial infarction.

In some embodiments, a cell proliferative disorder is a cancer of the liver, lung, spleen, pancreas, colon, skin, bladder, eye, brain, esophagus, head, neck, ovary, testes, prostate, the like or combination thereof. Non-limiting examples of cancers include hematopoietic neoplastic disorders, which are diseases involving hyperplastic/neoplastic cells of hematopoietic origin (e.g., arising from myeloid, lymphoid or erythroid lineages, or precursor cells thereof), and can arise from poorly differentiated acute leukemias (e.g., erythroblastic leukemia and acute megakaryoblastic leukemia). Certain myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)). Certain lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Certain forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. A cell proliferative disorder sometimes is a non-endocrine tumor or endocrine tumor. Illustrative examples of non-endocrine tumors include, but are not limited to, adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor sometimes is an islet cell tumor.

In some embodiments, a wasting disorder or degenerative disorder is cirrhosis, amyotrophic lateral sclerosis (ALS), Alzheimer's disease, Parkinson's disease, multiple system atrophy, atherosclerosis, progressive supranuclear palsy, Tay-Sachs disease, diabetes, heart disease, keratoconus, inflammatory bowel disease (IBD), prostatitis, osteoarthritis, osteoporosis, rheumatoid arthritis, Huntington's disease, chronic traumatic encephalopathy, chronic obstructive pulmonary disease (COPD), tuberculosis, chronic diarrhea, acquired immune deficiency syndrome (AIDS), superior mesenteric artery syndrome, the like or combination thereof.

In some embodiments, an autoimmune disorder is acute disseminated encephalomyelitis (ADEM), Addison's disease, alopecia greata, ankylosing spondylitis, antiphospholipid antibody syndrome (APS), autoimmune hemolytic anemia, autoimmune hepatitis, autoimmune inner ear disease, bullous pemphigoid, coeliac disease, Chagas disease, chronic obstructive pulmonary disease, Crohns Disease (a type of idiopathic inflammatory bowel disease “IBD”), dermatomyositis, diabetes mellitus type 1, endometriosis, Goodpasture's syndrome, Graves' disease, Guillain-Barrè syndrome (GBS), Hashimoto's disease, hidradenitis suppurativa, idiopathic thrombocytopenic purpura, interstitial cystitis, Lupus erythematosus, mixed connective tissue disease, morphea, multiple sclerosis (MS), myasthenia gravis, narcolepsy, euromyotonia, pemphigus vulgaris, pernicious anaemia, polymyositis, primary biliary cirrhosis, rheumatoid arthritis, schizophrenia, scleroderma, Sjögren's syndrome, temporal arteritis (also known as “giant cell arteritis”), ulcerative colitis (a type of idiopathic inflammatory bowel disease “IBD”), vasculitis, vitiligo, Wegener's granulomatosis, the like or combination thereof.

### Marker Detection and Quantification Technologies

Any suitable technology can be used to detect and/or quantify a marker (e.g., methylation state of a locus). Non-limiting examples of technologies that can be utilized to detect and/or quantify a marker include mass spectrometry, amplification (e.g., digital PCR, quantitative polymerase chain reaction (qPCR)), sequencing (e.g., nanopore sequencing, base extension sequencing (e.g., single base extension sequencing)), array hybridization (e.g., microarray hybridization; gene-chip analysis), flow cytometry, gel electrophoresis (e.g., capillary electrophoresis), cytofluorimetric analysis, fluorescence microscopy, confocal laser scanning microscopy, laser scanning cytometry, affinity chromatography, manual batch mode separation, electric field suspension, the like and combinations of the foregoing. Further detail is provided hereafter for certain marker detection and/or quantification technologies.

Mass Spectrometry

In some embodiments, mass spectrometry is used to detect and/or quantify nucleic acid fragments. Mass spectrometry methods typically are used to determine the mass of a molecule, such as a nucleic acid fragment. In some embodiments, mass spectrometry is used in conjunction with another detection, enrichment and/or separation method known in the art or described herein such as, for example, MassARRAY, primer extension (e.g., MASSEXTEND), probe extension, methods using mass modified probes and/or primers, and the like. The relative signal strength, e.g., mass peak on a spectra, for a particular nucleic acid fragment can indicate the relative population of the fragment species amongst other nucleic acids in the sample (see e.g., Jurinke et al. (2004) Mol. Biotechnol. 26, 147-164).

Mass spectrometry generally works by ionizing chemical compounds to generate charged molecules or molecule fragments and measuring their mass-to-charge ratios. A typical mass spectrometry procedure involves several steps, including (1) loading a sample onto a mass spectrometry instrument followed by vaporization, (2) ionization of the sample components by any one of a variety of methods (e.g., impacting with an electron beam), resulting in charged particles (ions), (3) separation of ions according to their mass-to-charge ratio in an analyzer by electromagnetic fields, (4) detection of ions (e.g., by a quantitative method), and (5) processing of ion signals into mass spectra.

Mass spectrometry methods are known, and include without limitation quadrupole mass spectrometry, ion trap mass spectrometry, time-of-flight mass spectrometry, gas chromatography mass spectrometry and tandem mass spectrometry can be used with a method described herein. Processes associated with mass spectrometry are generation of gas-phase ions derived from the sample, and measurement of ions. Movement of gas-phase ions can be precisely controlled using electromagnetic fields generated in the mass spectrometer, and movement of ions in these electromagnetic fields is proportional to the mass to charge ratio (m/z) of each ion, which forms the basis of measuring m/z and mass. Movement of ions in these electromagnetic fields allows for containment and focusing of the ions which accounts for high sensitivity of mass spectrometry. During the course of m/z measurement, ions are transmitted with high efficiency to particle detectors that record the arrival of these ions. The quantity of ions at each m/z is demonstrated by peaks on a graph where the x axis is m/z and the y axis is relative abundance. Different mass spectrometers have different levels of resolution (i.e., the ability to resolve peaks between ions closely related in mass). Resolution generally is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1.

Certain mass spectrometry methods can utilize various combinations of ion sources and mass analyzers which allows for flexibility in designing customized detection protocols. In some embodiments, mass spectrometers can be programmed to transmit all ions from the ion source into the mass spectrometer either sequentially or at the same time. In some embodiments, a mass spectrometer can be programmed to select ions of a particular mass for transmission into the mass spectrometer while blocking other ions.

Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Mass analyzers include, for example, a quadrupole mass filter, ion trap mass analyzer and time-of-flight mass analyzer.

An ion formation process generally is a starting point for mass spectrum analysis. Several ionization methods are available and the choice of ionization method depends on the sample used for analysis. For example, for the analysis of polypeptides a relatively gentle ionization procedure such as electrospray ionization (ESI) can be desirable. For ESI, a solution containing the sample is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization. Matrix-assisted laser desorption ionization (MALDI) is a method in which a laser pulse is used to strike a sample that has been crystallized in an UV-absorbing compound matrix (e.g., 2,5-dihydroxybenzoic acid, alpha-cyano-4-hydroxycinammic acid, 3-hydroxypicolinic acid (3-HPA), di-ammoniumcitrate (DAC) and combinations thereof). Other ionization procedures known in the art include, for example, plasma and glow discharge, plasma desorption ionization, resonance ionization, and secondary ionization.

A variety of mass analyzers are available that can be paired with different ion sources. Different mass analyzers have different advantages as known in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below. Ion mobility mass (IM) spectrometry is a gas-phase separation method. IM separates gas-phase ions based on their collision cross-section and can be coupled with time-of-flight (TOF) mass spectrometry. IM-MS methods are known in the art.

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying a rf-only field. This allows quadrupoles to function as a lens or focusing system in regions of the mass spectrometer where ion transmission is needed without mass filtering.

A quadrupole mass analyzer, as well as the other mass analyzers described herein, can be programmed to analyze a defined m/z or mass range. Since the desired mass range of nucleic acid fragment is known, in some instances, a mass spectrometer can be programmed to transmit ions of the projected correct mass range while excluding ions of a higher or lower mass range. The ability to select a mass range can decrease the background noise in the assay and thus increase the signal-to-noise ratio. Thus, in some instances, a mass spectrometer can accomplish a separation step as well as detection and identification of certain mass-distinguishable nucleic acid fragments.

Ion trap mass spectrometry utilizes an ion trap mass analyzer. Typically, fields are applied such that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis can be accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. Control of the number of ions can be accomplished by varying the time over which ions are injected into the trap.

Time-of-flight mass spectrometry utilizes a time-of-flight mass analyzer. Typically, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion.

Gas chromatography mass spectrometry often can a target in real-time. The gas chromatography (GC) portion of the system separates the chemical mixture into pulses of analyte and the mass spectrometer (MS) identifies and quantifies the analyte.

Tandem mass spectrometry can utilize combinations of the mass analyzers described above. Tandem mass spectrometers can use a first mass analyzer to separate ions according to their m/z in order to isolate an ion of interest for further analysis. The isolated ion of interest is then broken into fragment ions (called collisionally activated dissociation or collisionally induced dissociation) and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers. Other tandem systems are known in the art including reflectron-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans.

For quantification, controls may be used which can provide a signal in relation to the amount of the nucleic acid fragment, for example, that is present or is introduced. A control to allow conversion of relative mass signals into absolute quantities can be accomplished by addition of a known quantity of a mass tag or mass label to each sample before detection of the nucleic acid fragments. Any mass tag that does not interfere with detection of the fragments can be used for normalizing the mass signal. Such standards typically have separation properties that are different from those of any of the molecular tags in the sample, and could have the same or different mass signatures.

A separation step sometimes can be used to remove salts, enzymes, or other buffer components from the nucleic acid sample. Several methods well known in the art, such as chromatography, gel electrophoresis, or precipitation, can be used to clean up the sample. For example, size exclusion chromatography or affinity chromatography can be used to remove salt from a sample. The choice of separation method can depend on the amount of a sample. For example, when small amounts of sample are available or a miniaturized apparatus is used, a micro-affinity chromatography separation step can be used. In addition, whether a separation step is desired, and the choice of separation method, can depend on the detection method used. Salts sometimes can absorb energy from the laser in matrix-assisted laser desorption/ionization and result in lower ionization efficiency. Thus, the efficiency of matrix-assisted laser desorption/ionization and electrospray ionization sometimes can be improved by removing salts from a sample.

MASSEXTEND technology may be used in some embodiments. Generally, a primer hybridizes to sample nucleic acid at a sequence within or adjacent to a site of interest. The addition of a DNA polymerase, plus a mixture of nucleotides and terminators, allows extension of the primer through the site of interest, and generates a unique mass product. The resultant mass of the primer extension product is then analyzed (e.g., using mass spectrometry) and used to determine the sequence and/or identity of the site of interest.

Nanopores

In some embodiments, nucleic acid fragments are detected and/or quantified using a nanopore. A nanopore can be used to obtain nucleotide sequencing information for nucleic acid fragments. In some embodiments, nucleic acid fragments are detected and/or quantified using a nanopore without obtaining nucleotide sequences. A nanopore is a small hole or channel, typically of the order of 1 nanometer in diameter. Certain transmembrane cellular proteins can act as nanopores (e.g., alpha-hemolysin). Nanopores can be synthesized (e.g., using a silicon platform). Immersion of a nanopore in a conducting fluid and application of a potential across it results in a slight electrical current due to conduction of ions through the nanopore. The amount of current which flows is sensitive to the size of the nanopore. As a nucleic acid fragment passes through a nanopore, the nucleic acid molecule obstructs the nanopore to a certain degree and generates a change to the current. In some embodiments, the duration of current change as the nucleic acid fragment passes through the nanopore can be measured.

In some embodiments, nanopore technology can be used in a method described herein for obtaining nucleotide sequence information for nucleic acid fragments. Nanopore sequencing is a single-molecule sequencing technology whereby a single nucleic acid molecule (e.g. DNA) is sequenced directly as it passes through a nanopore. As described above, immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree and generates characteristic changes to the current. The amount of current which can pass through the nanopore at any given moment therefore varies depending on whether the nanopore is blocked by an A, a C, a G, a T, or sometimes methyl-C. The change in the current through the nanopore as the DNA molecule passes through the nanopore represents a direct reading of the DNA sequence. In some embodiments, a nanopore can be used to identify individual DNA bases as they pass through the nanopore in the correct order (e.g., International Patent Application No. WO2010/004265).

There are a number of ways that nanopores can be used to sequence nucleic acid molecules. In some embodiments, an exonuclease enzyme, such as a deoxyribonuclease, is used. In this case, the exonuclease enzyme is used to sequentially detach nucleotides from a nucleic acid (e.g. DNA) molecule. The nucleotides are then detected and discriminated by the nanopore in order of their release, thus reading the sequence of the original strand. For such an embodiment, the exonuclease enzyme can be attached to the nanopore such that a proportion of the nucleotides released from the DNA molecule is capable of entering and interacting with the channel of the nanopore. The exonuclease can be attached to the nanopore structure at a site in close proximity to the part of the nanopore that forms the opening of the channel. In some embodiments, the exonuclease enzyme can be attached to the nanopore structure such that its nucleotide exit trajectory site is orientated towards the part of the nanopore that forms part of the opening.

In some embodiments, nanopore sequencing of nucleic acids involves the use of an enzyme that pushes or pulls the nucleic acid (e.g. DNA) molecule through the pore. In this case, the ionic current fluctuates as a nucleotide in the DNA molecule passes through the pore. The fluctuations in the current are indicative of the DNA sequence. For such an embodiment, the enzyme can be attached to the nanopore structure such that it is capable of pushing or pulling the target nucleic acid through the channel of a nanopore without interfering with the flow of ionic current through the pore. The enzyme can be attached to the nanopore structure at a site in close proximity to the part of the structure that forms part of the opening. The enzyme can be attached to the subunit, for example, such that its active site is orientated towards the part of the structure that forms part of the opening.

In some embodiments, nanopore sequencing of nucleic acids involves detection of polymerase bi-products in close proximity to a nanopore detector. In this case, nucleoside phosphates (nucleotides) are labeled so that a phosphate labeled species is released upon the addition of a polymerase to the nucleotide strand and the phosphate labeled species is detected by the pore. Typically, the phosphate species contains a specific label for each nucleotide. As nucleotides are sequentially added to the nucleic acid strand, the bi-products of the base addition are detected. The order that the phosphate labeled species are detected can be used to determine the sequence of the nucleic acid strand.

Probes

In some embodiments, nucleic acid fragments are detected and/or quantified using one or more probes. In some embodiments, quantification comprises quantifying target nucleic acid specifically hybridized to the probe. In some embodiments, quantification comprises quantifying the probe in the hybridization product. In some embodiments, quantification comprises quantifying target nucleic acid specifically hybridized to the probe and quantifying the probe in the hybridization product. In some embodiments, quantification comprises quantifying the probe after dissociating from the hybridization product. Quantification of hybridization product, probe and/or nucleic acid target can comprise use of, for example, mass spectrometry, MASSARRAY and/or MASSEXTEND technology, as described herein.

In some embodiments, probes are designed such that they each hybridize to a nucleic acid of interest in a sample. For example, a probe may comprise a polynucleotide sequence that is complementary to a nucleic acid of interest or may comprise a series of monomers that can bind to a nucleic acid of interest. Probes may be any length suitable to hybridize (e.g., completely hybridize) to one or more nucleic acid fragments of interest. For example, probes may be of any length which spans or extends beyond the length of a nucleic acid fragment to which it hybridizes. Probes may be about 10 bp or more in length. For example, probes may be at least about 20, 30, 40, 50, 60, 70, 80, 100, 200, 300, 400, 500, 600, 700, 800, 900 or 1000 bp in length. In some embodiments, a detection and/or quantification method is used to detect and/or quantify probe-nucleic acid fragment duplexes.

Probes may be designed and synthesized according to methods known in the art and described herein for oligonucleotides (e.g., capture oligonucleotides). Probes also may include any of the properties known in the art and described herein for oligonucleotides. Probes herein may be designed such that they comprise nucleotides (e.g., adenine (A), thymine (T), cytosine (C), guanine (G) and uracil (U)), modified nucleotides (e.g., mass-modified nucleotides, pseudouridine, dihydrouridine, inosine (I), and 7-methylguanosine), synthetic nucleotides, degenerate bases (e.g., 6H,8H-3,4-dihydropyrimido[4,5-c][1,2]oxazin-7-one (P), 2-amino-6-methoxyaminopurine (K), N6-methoxyadenine (Z), and hypoxanthine (I)), universal bases and/or monomers other than nucleotides, modified nucleotides or synthetic nucleotides, mass tags or combinations thereof.

In some embodiments, probes are dissociated (i.e., separated) from their corresponding nucleic acid fragments. Probes may be separated from their corresponding nucleic acid fragments using any method known in the art, including, but not limited to, heat denaturation. Probes can be distinguished from corresponding nucleic acid fragments by a method known in the art or described herein for labeling and/or isolating a species of molecule in a mixture. For example, a probe and/or nucleic acid fragment may comprise a detectable property such that a probe is distinguishable from the nucleic acid to which it hybridizes. Non-limiting examples of detectable properties include mass properties, optical properties, electrical properties, magnetic properties, chemical properties, and time and/or speed through an opening of known size. In some embodiments, probes and sample nucleic acid fragments are physically separated from each other, Separation can be accomplished, for example, using capture ligands, such as biotin or other affinity ligands, and capture agents, such as avidin, streptavidin, an antibody, or a receptor. A probe or nucleic acid fragment can contain a capture ligand having specific binding activity for a capture agent. For example, fragments from a nucleic acid sample can be biotinylated or attached to an affinity ligand using methods well known in the art and separated away from the probes using a pull-down assay with steptavidin-coated beads, for example. In some embodiments, a capture ligand and capture agent or any other moiety (e.g., mass tag) can be used to add mass to the nucleic acid fragments such that they can be excluded from the mass range of the probes detected in a mass spectrometer. In some embodiments, mass is added to the probes, addition of a mass tag for example, to shift the mass range away from the mass range for the nucleic acid fragments. In some embodiments, a detection and/or quantification method is used to detect and/or quantify dissociated nucleic acid fragments. In some embodiments, detection and/or quantification method is used to detect and/or quantify dissociated probes.

Digital PCR

In some embodiments, nucleic acid fragments are detected and/or quantified using digital PCR technology. Digital polymerase chain reaction (digital PCR or dPCR) can be used, for example, to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number.

Nucleic Acid Sequencing

In some embodiments, nucleic acids (e.g., nucleic acid fragments, sample nucleic acid, circulating cell-free nucleic acid) may be sequenced. In some embodiments, a full or substantially full sequence is obtained and sometimes a partial sequence is obtained. In some embodiments, a nucleic acid is not sequenced, and the sequence of a nucleic acid is not determined by a sequencing method, when performing a method described herein. Sequencing, mapping and related analytical methods are known in the art (e.g., United States Patent Application Publication US2009/0029377, incorporated by reference). Certain aspects of such processes are described hereafter.

Certain sequencing technologies generate nucleotide sequence reads. As used herein, “reads” (i.e., “a read”, “a sequence read”) are short nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads).

In some embodiments the nominal, average, mean or absolute length of single-end reads sometimes is about 20 contiguous nucleotides to about 50 contiguous nucleotides, sometimes about 30 contiguous nucleotides to about 40 contiguous nucleotides, and sometimes about 35 contiguous nucleotides or about 36 contiguous nucleotides. In some embodiments, the nominal, average, mean or absolute length of single-end reads is about 20 to about 30 bases in length. In some embodiments, the nominal, average, mean or absolute length of single-end reads is about 24 to about 28 bases in length. In some embodiments, the nominal, average, mean or absolute length of single-end reads is about 21, 22, 23, 24, 25, 26, 27, 28 or about 29 bases in length.

In certain embodiments, the nominal, average, mean or absolute length of the paired-end reads sometimes is about 10 contiguous nucleotides to about 50 contiguous nucleotides (e.g., about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleotides in length), sometimes is about 15 contiguous nucleotides to about 25 contiguous nucleotides, and sometimes is about 17 contiguous nucleotides, about 18 contiguous nucleotides, about 20 contiguous nucleotides, about 25 contiguous nucleotides, about 36 contiguous nucleotides or about 45 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from the blood of a pregnant female can be reads from a mixture of fetal and maternal nucleic acid. A mixture of relatively short reads can be transformed by processes described herein into a representation of a genomic nucleic acid present in the pregnant female and/or in the fetus. A mixture of relatively short reads can be transformed into a representation of a copy number variation (e.g., a maternal and/or fetal copy number variation), genetic variation or an aneuploidy, for example. Reads of a mixture of maternal and fetal nucleic acid can be transformed into a representation of a composite chromosome or a segment thereof comprising features of one or both maternal and fetal chromosomes. In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

Sequence reads can be mapped and the number of reads or sequence tags mapping to a specified nucleic acid region (e.g., a chromosome, a bin, a genomic section) are referred to as counts. In some embodiments, counts can be manipulated or transformed (e.g., normalized, combined, added, filtered, selected, averaged, derived as a mean, the like, or a combination thereof). In some embodiments, counts can be transformed to produce normalized counts. Normalized counts for multiple genomic sections can be provided in a profile (e.g., a genomic profile, a chromosome profile, a profile of a segment of a chromosome). One or more different elevations in a profile also can be manipulated or transformed (e.g., counts associated with elevations can be normalized) and elevations can be adjusted.

In some embodiments, one nucleic acid sample from one individual is sequenced. In certain embodiments, nucleic acid samples from two or more biological samples, where each biological sample is from one individual or two or more individuals, are pooled and the pool is sequenced. In the latter embodiments, a nucleic acid sample from each biological sample often is identified by one or more unique identification tags.

In some embodiments, a fraction of the genome is sequenced, which sometimes is expressed in the amount of the genome covered by the determined nucleotide sequences (e.g., “fold” coverage less than 1). When a genome is sequenced with about 1-fold coverage, roughly 100% of the nucleotide sequence of the genome is represented by reads. A genome also can be sequenced with redundancy, where a given region of the genome can be covered by two or more reads or overlapping reads (e.g., “fold” coverage greater than 1). In some embodiments, a genome is sequenced with about 0.01-fold to about 100-fold coverage, about 0.2-fold to 20-fold coverage, or about 0.2-fold to about 1-fold coverage (e.g., about 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage).

In certain embodiments, a subset of nucleic acid fragments is selected prior to sequencing. In certain embodiments, hybridization-based techniques (e.g., using oligonucleotide arrays) can be used to first select for nucleic acid sequences from certain chromosomes (e.g., a potentially aneuploid chromosome and other chromosome(s) not involved in the aneuploidy tested) or a segment thereof (e.g., a sub-chromosomal region). In some embodiments, nucleic acid can be fractionated by size (e.g., by gel electrophoresis, size exclusion chromatography or by microfluidics-based approach) and in certain instances, fetal nucleic acid can be enriched by selecting for nucleic acid having a lower molecular weight (e.g., less than 300 base pairs, less than 200 base pairs, less than 150 base pairs, less than 100 base pairs). In some embodiments, fetal nucleic acid can be enriched by suppressing maternal background nucleic acid, such as by the addition of formaldehyde. In some embodiments, a portion or subset of a pre-selected set of nucleic acid fragments is sequenced randomly. In some embodiments, the nucleic acid is amplified prior to sequencing. In some embodiments, a portion or subset of the nucleic acid is amplified prior to sequencing.

In some embodiments, a sequencing library is prepared prior to or during a sequencing process. Methods for preparing a sequencing library are known in the art and commercially available platforms may be used for certain applications. Certain commercially available library platforms may be compatible with certain nucleotide sequencing processes described herein. For example, one or more commercially available library platforms may be compatible with a sequencing by synthesis process. In some embodiments, a ligation-based library preparation method is used (e.g., ILLUMINA TRUSEQ, Illumina, San Diego Calif.). Ligation-based library preparation methods typically use a methylated adaptor design which can incorporate an index sequence at the initial ligation step and often can be used to prepare samples for single-read sequencing, paired-end sequencing and multiplexed sequencing. In some embodiments, a transposon-based library preparation method is used (e.g., EPICENTRE NEXTERA, Illumina, Inc., California). Transposon-based methods typically use in vitro transposition to simultaneously fragment and tag DNA in a single-tube reaction (often allowing incorporation of platform-specific tags and optional barcodes), and prepare sequencer-ready libraries.

Any sequencing method suitable for conducting methods described herein can be utilized. In some embodiments, a high-throughput sequencing method is used. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin Chem 55:641-658 (2009)). Such sequencing methods also can provide digital quantitative information, where each sequence read is a countable “sequence tag” or “count” representing an individual clonal DNA template, a single DNA molecule, bin or chromosome. Next generation sequencing techniques capable of sequencing DNA in a massively parallel fashion are collectively referred to herein as “massively parallel sequencing” (MPS). Certain MPS techniques include a sequencing-by-synthesis process. High-throughput sequencing technologies include, for example, sequencing-by-synthesis with reversible dye terminators, sequencing by oligonucleotide probe ligation, pyrosequencing and real time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single molecule sequencing, single molecule real time (SMRT) sequencing, nanopore sequencing, ION Torrent and RNA polymerase (RNAP) sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in a method provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

A nucleic acid sequencing technology that may be used in a method described herein is sequencing-by-synthesis and reversible terminator-based sequencing (e.g. Illumina's Genome Analyzer; Genome Analyzer II; HISEQ 2000; HISEQ 2500 (IIlumina, San Diego Calif.)). With this technology, millions of nucleic acid (e.g. DNA) fragments can be sequenced in parallel. In one example of this type of sequencing technology, a flow cell is used which contains an optically transparent slide with 8 individual lanes on the surfaces of which are bound oligonucleotide anchors (e.g., adaptor primers). A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs.

In certain sequencing by synthesis procedures, for example, template DNA (e.g., circulating cell-free DNA (ccfDNA)) sometimes can be fragmented into lengths of several hundred base pairs in preparation for library generation. In some embodiments, library preparation can be performed without further fragmentation or size selection of the template DNA (e.g., ccfDNA). Sample isolation and library generation may be performed using automated methods and apparatus, in certain embodiments. Briefly, template DNA is end repaired by a fill-in reaction, exonuclease reaction or a combination of a fill-in reaction and exonuclease reaction. The resulting blunt-end repaired template DNA is extended by a single nucleotide, which is complementary to a single nucleotide overhang on the 3′ end of an adapter primer, and often increases ligation efficiency. Any complementary nucleotides can be used for the extension/overhang nucleotides (e.g., A/T, C/G), however adenine frequently is used to extend the end-repaired DNA, and thymine often is used as the 3′ end overhang nucleotide.

In certain sequencing by synthesis procedures, for example, adapter oligonucleotides are complementary to the flow-cell anchors, and sometimes are utilized to associate the modified template DNA (e.g., end-repaired and single nucleotide extended) with a solid support, such as the inside surface of a flow cell, for example. In some embodiments, the adapter also includes identifiers (i.e., indexing nucleotides, or “barcode” nucleotides (e.g., a unique sequence of nucleotides usable as an identifier to allow unambiguous identification of a sample and/or chromosome)), one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Identifiers or nucleotides contained in an adapter often are six or more nucleotides in length, and frequently are positioned in the adaptor such that the identifier nucleotides are the first nucleotides sequenced during the sequencing reaction. In certain embodiments, identifier nucleotides are associated with a sample but are sequenced in a separate sequencing reaction to avoid compromising the quality of sequence reads. Subsequently, the reads from the identifier sequencing and the DNA template sequencing are linked together and the reads de-multiplexed. After linking and de-multiplexing the sequence reads and/or identifiers can be further adjusted or processed as described herein.

In certain sequencing by synthesis procedures, utilization of identifiers allows multiplexing of sequence reactions in a flow cell lane, thereby allowing analysis of multiple samples per flow cell lane. The number of samples that can be analyzed in a given flow cell lane often is dependent on the number of unique identifiers utilized during library preparation and/or probe design. Non limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively). A method described herein can be performed using any number of unique identifiers (e.g., 4, 8, 12, 24, 48, 96, or more). The greater the number of unique identifiers, the greater the number of samples and/or chromosomes, for example, that can be multiplexed in a single flow cell lane. Multiplexing using 12 identifiers, for example, allows simultaneous analysis of 96 samples (e.g., equal to the number of wells in a 96 well microwell plate) in an 8 lane flow cell. Similarly, multiplexing using 48 identifiers, for example, allows simultaneous analysis of 384 samples (e.g., equal to the number of wells in a 384 well microwell plate) in an 8 lane flow cell.

In certain sequencing by synthesis procedures, adapter-modified, single-stranded template DNA is added to the flow cell and immobilized by hybridization to the anchors under limiting-dilution conditions. In contrast to emulsion PCR, DNA templates are amplified in the flow cell by “bridge” amplification, which relies on captured DNA strands “arching” over and hybridizing to an adjacent anchor oligonucleotide. Multiple amplification cycles convert the single-molecule DNA template to a clonally amplified arching “cluster,” with each cluster containing approximately 1000 clonal molecules. Approximately 1×10̂9 separate clusters can be generated per flow cell. For sequencing, the clusters are denatured, and a subsequent chemical cleavage reaction and wash leave only forward strands for single-end sequencing. Sequencing of the forward strands is initiated by hybridizing a primer complementary to the adapter sequences, which is followed by addition of polymerase and a mixture of four differently colored fluorescent reversible dye terminators. The terminators are incorporated according to sequence complementarity in each strand in a clonal cluster. After incorporation, excess reagents are washed away, the clusters are optically interrogated, and the fluorescence is recorded. With successive chemical steps, the reversible dye terminators are unblocked, the fluorescent labels are cleaved and washed away, and the next sequencing cycle is performed. This iterative, sequencing-by-synthesis process sometimes requires approximately 2.5 days to generate read lengths of 36 bases. With 50×106 clusters per flow cell, the overall sequence output can be greater than 1 billion base pairs (Gb) per analytical run.

Another nucleic acid sequencing technology that may be used with a method described herein is 454 sequencing (Roche). 454 sequencing uses a large-scale parallel pyrosequencing system capable of sequencing about 400-600 megabases of DNA per run. The process typically involves two steps. In the first step, sample nucleic acid (e.g. DNA) is sometimes fractionated into smaller fragments (300-800 base pairs) and polished (made blunt at each end). Short adaptors are then ligated onto the ends of the fragments. These adaptors provide priming sequences for both amplification and sequencing of the sample-library fragments. One adaptor (Adaptor B) contains a 5′-biotin tag for immobilization of the DNA library onto streptavidin-coated beads. After nick repair, the non-biotinylated strand is released and used as a single-stranded template DNA (sstDNA) library. The sstDNA library is assessed for its quality and the optimal amount (DNA copies per bead) needed for emPCR is determined by titration. The sstDNA library is immobilized onto beads. The beads containing a library fragment carry a single sstDNA molecule. The bead-bound library is emulsified with the amplification reagents in a water-in-oil mixture. Each bead is captured within its own microreactor where PCR amplification occurs. This results in bead-immobilized, clonally amplified DNA fragments.

In the second step of 454 sequencing, single-stranded template DNA library beads are added to an incubation mix containing DNA polymerase and are layered with beads containing sulfurylase and luciferase onto a device containing pico-liter sized wells. Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing exploits the release of pyrophosphate (PPi) upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed (see, for example, Margulies, M. et al. Nature 437:376-380 (2005)).

Another nucleic acid sequencing technology that may be used in a method provided herein is Applied Biosystems' SOLiD™ technology. In SOLiD™ sequencing-by-ligation, a library of nucleic acid fragments is prepared from the sample and is used to prepare clonal bead populations. With this method, one species of nucleic acid fragment will be present on the surface of each bead (e.g. magnetic bead). Sample nucleic acid (e.g. genomic DNA) is sheared into fragments, and adaptors are subsequently attached to the 5′ and 3′ ends of the fragments to generate a fragment library. The adapters are typically universal adapter sequences so that the starting sequence of every fragment is both known and identical. Emulsion PCR takes place in microreactors containing all the necessary reagents for PCR. The resulting PCR products attached to the beads are then covalently bound to a glass slide. Primers then hybridize to the adapter sequence within the library template. A set of four fluorescently labeled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. Following a series of ligation cycles, the extension product is removed and the template is reset with a primer complementary to the n−1 position for a second round of ligation cycles. Often, five rounds of primer reset are completed for each sequence tag. Through the primer reset process, each base is interrogated in two independent ligation reactions by two different primers. For example, the base at read position 5 is assayed by primer number 2 in ligation cycle 2 and by primer number 3 in ligation cycle 1.

Another nucleic acid sequencing technology that may be used in a method described herein is Helicos True Single Molecule Sequencing (tSMS). In the tSMS technique, a polyA sequence is added to the 3′ end of each nucleic acid (e.g. DNA) strand from the sample. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm2. The flow cell is then loaded into a sequencing apparatus and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step (see, for example, Harris T. D. et al., Science 320:106-109 (2008)).

Another nucleic acid sequencing technology that may be used in a method provided herein is the single molecule, real-time (SMRT™) sequencing technology of Pacific Biosciences. With this method, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is then repeated.

Another nucleic acid sequencing technology that may be used in a method described herein is ION TORRENT (Life Technologies) single molecule sequencing which pairs semiconductor technology with a simple sequencing chemistry to directly translate chemically encoded information (A, C, G, T) into digital information (0, 1) on a semiconductor chip. ION TORRENT uses a high-density array of micro-machined wells to perform nucleic acid sequencing in a massively parallel way. Each well holds a different DNA molecule. Beneath the wells is an ion-sensitive layer and beneath that an ion sensor. Typically, when a nucleotide is incorporated into a strand of DNA by a polymerase, a hydrogen ion is released as a byproduct. If a nucleotide, for example a C, is added to a DNA template and is then incorporated into a strand of DNA, a hydrogen ion will be released. The charge from that ion will change the pH of the solution, which can be detected by an ion sensor. A sequencer can call the base, going directly from chemical information to digital information. The sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection (i.e. detection without scanning, cameras or light), each nucleotide incorporation is recorded in seconds.

Another nucleic acid sequencing technology that may be used in a method described herein is the chemical-sensitive field effect transistor (CHEMFET) array. In one example of this sequencing technique, DNA molecules are placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a CHEMFET sensor. An array can have multiple CHEMFET sensors. In another example, single nucleic acids are attached to beads, and the nucleic acids can be amplified on the bead, and the individual beads can be transferred to individual reaction chambers on a CHEMFET array, with each chamber having a CHEMFET sensor, and the nucleic acids can be sequenced (see, for example, U.S. Patent Application Publication No. 2009/0026082).

Another nucleic acid sequencing technology that may be used in a method described herein is electron microscopy. In one example of this sequencing technique, individual nucleic acid (e.g. DNA) molecules are labeled using metallic labels that are distinguishable using an electron microscope. These molecules are then stretched on a flat surface and imaged using an electron microscope to measure sequences (see, for example, Moudrianakis E. N. and Beer M. Proc Natl Acad Sci USA. 1965 March; 53:564-71). In some embodiments, transmission electron microscopy (TEM) is used (e.g. Halcyon Molecular's TEM method). This method, termed Individual Molecule Placement Rapid Nano Transfer (IMPRNT), includes utilizing single atom resolution transmission electron microscope imaging of high-molecular weight (e.g. about 150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA (see, for example, International Patent Application No. WO 2009/046445).

Other sequencing methods that may be used to conduct methods herein include digital PCR and sequencing by hybridization. Digital polymerase chain reaction (digital PCR or dPCR) can be used to directly identify and quantify nucleic acids in a sample. Digital PCR can be performed in an emulsion, in some embodiments. For example, individual nucleic acids are separated, e.g., in a microfluidic chamber device, and each nucleic acid is individually amplified by PCR. Nucleic acids can be separated such that there is no more than one nucleic acid per well. In some embodiments, different probes can be used to distinguish various alleles (e.g. fetal alleles and maternal alleles). Alleles can be enumerated to determine copy number. In sequencing by hybridization, the method involves contacting a plurality of polynucleotide sequences with a plurality of polynucleotide probes, where each of the plurality of polynucleotide probes can be optionally tethered to a substrate. The substrate can be a flat surface with an array of known nucleotide sequences, in some embodiments. The pattern of hybridization to the array can be used to determine the polynucleotide sequences present in the sample. In some embodiments, each probe is tethered to a bead, e.g., a magnetic bead or the like. Hybridization to the beads can be identified and used to identify the plurality of polynucleotide sequences within the sample.

In some embodiments, chromosome-specific sequencing is performed. In some embodiments, chromosome-specific sequencing is performed utilizing DANSR (digital analysis of selected regions). Digital analysis of selected regions enables simultaneous quantification of hundreds of loci by cfDNA-dependent catenation of two locus-specific oligonucleotides via an intervening ‘bridge’ oligo to form a PCR template. In some embodiments, chromosome-specific sequencing is performed by generating a library enriched in chromosome-specific sequences. In some embodiments, sequence reads are obtained only for a selected set of chromosomes. In some embodiments, sequence reads are obtained only for chromosomes 21, 18 and 13.

The length of the sequence read often is associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median, mode or average length of about 4 bp to 900 bp long (e.g. about 5 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median, mode or average length of about 1,000 bp or more.

Methylation-Sensitive Detection and Quantification Technologies

Non-limiting examples of processes for detecting and/or quantifying a methylation state of a marker are described in International Application Publication No. WO 2011/034631 published on Mar. 24, 2011 (International Application No. PCT/US2010/027879 filed on Mar. 18, 2010) and in International Application Publication No. WO 2012/149339 published on Nov. 1, 2012 (International Application No. PCT/US2012/035479 filed on Apr. 27, 2012). In some embodiments, a methylation sensitive procedure is utilized as part of detecting and/or quantifying a marker. Non-limiting examples of methylation sensitive procedures include bisulfite treatment of DNA, bisulfite sequencing, methylation specific PCR (MSP), quantitative methylation specific PCR (QPSP), combined bisulfite restriction analysis (COBRA), methylation-sensitive single nucleotide primer extension (Ms-SNuPE), MethylLight, methylation pyrosequencing, immunoprecipitation with 5-Methyl Cytosine (MeDIP), Methyl CpG Immunoprecipitation (MCIp; e.g., use of an antibody that specifically binds to a methyl-CpG binding domain (MBD) of a MBD2 methyl binding protein (MBD-Fc) for immunoprecipitation of methylated or unmethylated DNA), and methyl-dependent enzyme digestion with McrBC.

### Marker Enrichment

A process for detecting and/or quantifying a marker sometimes includes enriching for nucleic acid comprising one or more markers. Certain marker enrichment processes are described herein, and nucleic acid containing one or markers often is referred to as “target nucleic acid.”

In some embodiments, nucleic acid (e.g., ccfNA) is enriched or relatively enriched for a subpopulation or species of nucleic acid. Nucleic acid subpopulations can include, for example, nucleic acid comprising one or more markers and nucleic acid not containing a marker. In some embodiments, nucleic acid is enriched for fragments comprising certain nucleic acid sequences (e.g., marker sequences). Such enriched samples can be used in conjunction with a method provided herein. Thus, in certain embodiments, a method described herein may include an additional step of enriching for a subpopulation of nucleic acid in a sample. In certain embodiments, nucleic acid not containing a marker is selectively removed (partially, substantially, almost completely or completely removed) from sample nucleic acid. In some embodiments, enriching for a particular low copy number species nucleic acid (e.g., marker nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, all of which are incorporated by reference herein.

Certain enrichment methods exploit epigenetic differences between polynucleotides. Methylation-based fetal nucleic acid enrichment methods are described in U.S. Patent Application Publication No. 2010/0105049. Such methods sometimes involve binding a sample nucleic acid to a methylation-specific binding agent (methyl-CpG binding protein (MBD), methylation specific antibodies, and the like) and separating bound nucleic acid from unbound nucleic acid based on differential methylation status. Such methods also can include the use of methylation-sensitive restriction enzymes (as described above; e.g., Hhal and HpaII), which allow for the enrichment of marker regions in a sample by selectively digesting sample nucleic acid with an enzyme that selectively, and completely or substantially, digests the nucleic acid to enrich the sample for at least one marker polynucleotide.

Some enrichment methods include a restriction endonuclease enhanced polymorphic sequence approach, such as a method described in U.S. Patent Application Publication No. 2009/0317818, which is incorporated by reference herein. Such methods include cleavage of nucleic acid comprising a non-target allele with a restriction endonuclease that recognizes the nucleic acid comprising the non-target allele but not the target allele; and amplification of non-cleaved nucleic acid but not cleaved nucleic acid, where the non-cleaved, amplified nucleic acid represents enriched target nucleic acid (e.g., nucleic acid comprising a marker) relative to non-target nucleic acid (e.g., nucleic acid not containing a marker). In some embodiments, nucleic acid may be selected such that it comprises an allele having a polymorphic site that is susceptible to selective digestion by a cleavage agent, for example.

Certain enrichment methods include selective enzymatic degradation approaches. Such methods involve protecting target sequences from exonuclease digestion thereby facilitating the elimination in a sample of non-target sequences (e.g., non-marker sequences). For example, in one approach, sample nucleic acid is denatured to generate single stranded nucleic acid, single stranded nucleic acid is contacted with at least one target-specific primer pair under suitable annealing conditions, annealed primers are extended by nucleotide polymerization generating double stranded target sequences, and digesting single stranded nucleic acid using a nuclease that digests single stranded (i.e. non-target) nucleic acid. In some embodiments, the method can be repeated for at least one additional cycle. In some embodiments, the same target-specific primer pair is used to prime each of the first and second cycles of extension, and sometimes different target-specific primer pairs are used for the first and second cycles.

Some methods for enriching for a nucleic acid subpopulation (e.g., target nucleic acid) that can be used with a method described herein include massively parallel signature sequencing (MPSS) approaches. MPSS typically is a solid phase method that uses adapter (i.e. tag) ligation, followed by adapter decoding, and reading of the nucleic acid sequence in small increments. Tagged PCR products are typically amplified such that each nucleic acid generates a PCR product with a unique tag. Tags are often used to attach the PCR products to microbeads. After several rounds of ligation-based sequence determination, for example, a sequence signature can be identified from each bead. Each signature sequence (MPSS tag) in a MPSS dataset is analyzed, compared with all other signatures, and all identical signatures are counted.

Certain MPSS-based enrichment methods can include amplification-based approaches (e.g., PCR amplification). In some embodiments, loci-specific amplification methods can be used (e.g., using loci-specific amplification primers, such as, for example, primers designed to amplify marker sequences). In some embodiments, a multiplex SNP allele PCR approach can be used. In some embodiments, a multiplex SNP allele PCR approach can be used in combination with uniplex sequencing. For example, such an approach can involve the use of multiplex PCR (e.g., MASSARRAY system) and incorporation of capture probe sequences into amplicons followed by sequencing using, for example, the Illumina MPSS system. In some embodiments, a multiplex SNP allele PCR approach can be used in combination with a three-primer system and indexed sequencing. For example, such an approach can involve the use of multiplex PCR (e.g., MASSARRAY system) with primers having a first capture probe incorporated into certain loci-specific forward PCR primers and adapter sequences incorporated into loci-specific reverse PCR primers, to thereby generate amplicons, followed by a secondary PCR to incorporate reverse capture sequences and molecular index barcodes for sequencing using, for example, the Illumina MPSS system. In some embodiments, a multiplex SNP allele PCR approach can be used in combination with a four-primer system and indexed sequencing. For example, such an approach can involve the use of multiplex PCR (e.g., MASSARRAY system) with primers having adaptor sequences incorporated into both loci-specific forward and loci-specific reverse PCR primers, followed by a secondary PCR to incorporate both forward and reverse capture sequences and molecular index barcodes for sequencing (e.g., using an Illumina MPSS system). In some embodiments, a microfluidics approach can be used, and sometimes an array-based microfluidics approach can be used for such processes. For example, such an approach can involve the use of a microfluidics array (e.g., Fluidigm) for amplification at low plex and incorporation of index and capture probes, followed by sequencing. In some embodiments, an emulsion microfluidics approach can be used, such as, for example, digital droplet PCR.

In some instances, universal amplification methods can be used (e.g., using universal or non-loci-specific amplification primers). In some embodiments, universal amplification methods can be used in combination with pull-down approaches. In some embodiments, a method can include biotinylated ultramer pull-down (e.g., biotinylated pull-down assays from Agilent or IDT) from a universally amplified sequencing library. For example, such an approach can involve preparation of a standard library, enrichment for selected regions by a pull-down assay, and a secondary universal amplification step. In some embodiments, pull-down approaches can be used in combination with ligation-based methods. In some embodiments, a method can include biotinylated ultramer pull down with sequence specific adapter ligation (e.g., HALOPLEX PCR, Halo Genomics). For example, such an approach can involve the use of selector probes to capture restriction enzyme-digested fragments, followed by ligation of captured products to an adaptor, and universal amplification followed by sequencing. In some embodiments, pull-down approaches can be used in combination with extension and ligation-based methods. In some embodiments, a method can include molecular inversion probe (MIP) extension and ligation. For example, such an approach can involve the use of molecular inversion probes in combination with sequence adapters followed by universal amplification and sequencing. In some embodiments, complementary DNA can be synthesized and sequenced without amplification.

In some instances, extension and ligation approaches can be performed without a pull-down component. In some embodiments, a method can include loci-specific forward and reverse primer hybridization, extension and ligation. Such methods can further include universal amplification or complementary DNA synthesis without amplification, followed by sequencing. At times, such methods can reduce or exclude background sequences during analysis.

In some instances, pull-down approaches can be used with an optional amplification component or with no amplification component. In some embodiments, a method can include a modified pull-down assay and ligation with full incorporation of capture probes without universal amplification. For example, such an approach can involve the use of modified selector probes to capture restriction enzyme-digested fragments, followed by ligation of captured products to an adaptor, optional amplification, and sequencing. In some embodiments, a method can include a biotinylated pull-down assay with extension and ligation of adaptor sequence in combination with circular single stranded ligation. For example, such an approach can involve the use of selector probes to capture regions of interest (i.e. target sequences), extension of the probes, adaptor ligation, single stranded circular ligation, optional amplification, and sequencing. In some embodiments, the analysis of the sequencing result can separate target sequences from background.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). Length-based separation approaches can include fragment circularization, chemical treatment (e.g., formaldehyde, polyethylene glycol (PEG)), mass spectrometry and/or size-specific nucleic acid amplification, for example.

Certain length-based separation methods that can be used with methods described herein employ a selective sequence tagging approach, for example. The term “sequence tagging” refers to incorporating a recognizable and distinct sequence into a nucleic acid or population of nucleic acids. The term “sequence tagging” as used herein has a different meaning than the term “sequence tag” described later herein. In such sequence tagging methods, a fragment size species (e.g., short fragments) nucleic acids are subjected to selective sequence tagging in a sample that includes long and short nucleic acids. Such methods typically involve performing a nucleic acid amplification reaction using a set of nested primers which include inner primers and outer primers. In some embodiments, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms, which include, for example, blocked extension of the inner primers by the prior annealing and extension of the outer primers. Enrichment for tagged fragments can be accomplished by any of a variety of methods, including for example, exonuclease digestion of single stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another length-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

### Nucleic Acid Separation

In some embodiments, a marker detection and/or quantification process includes a nucleic acid separation process. In some embodiments, nucleic acid is enriched for fragments from a select genomic region (e.g., region containing one or more markers) using one or more sequence-based separation methods described herein and/or known in the art. In some embodiments, nucleic acid is enriched for sequences or fragments comprising one or more select nucleotide sequences (e.g., marker sequences) using one or more sequence-based separation methods described herein and/or known in the art. In some embodiments, separating nucleic acid comprises contacting nucleic acid with a hybridization probe under conditions in which nucleic acid comprising a marker sequence specifically hybridizes to the probe. In some embodiments, the probe is in an array. In some embodiments, separated nucleic acid is quantified using a quantification method described herein.

Sequence-based separation generally is based on nucleotide sequences present in fragments of interest (e.g., target sequences (e.g., marker sequences) and/or reference sequences) and substantially not present in other fragments of a sample. In some embodiments, sequence-based separation can generate separated target fragments and/or separated reference fragments, Separated target fragments and/or separated reference fragments typically are isolated away from remaining fragments in the nucleic acid sample. In some embodiments, separated target fragments and the separated reference fragments also are isolated away from each other (e.g., isolated in separate assay compartments). In some embodiments, separated target fragments and separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from the nucleic acid sample. Commercially available nucleic acid capture systems include, for example, Nimblegen sequence capture system (Roche NimbleGen, Madison, Wis.); Illumina BEADARRAY platform (Illumina, San Diego, Calif.); Affymetrix GENECHIP platform (Affymetrix, Santa Clara, Calif.); Agilent SureSelect Target Enrichment System (Agilent Technologies, Santa Clara, Calif.); and related platforms. Such methods typically involve hybridization of a capture oligonucleotide to a portion or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci (e.g., one of chromosomes 21, 18, 13, or X or a reference chromosome). In some embodiments, capture oligonucleotides are selected or designed such that they preferentially hybridize to nucleic acid fragments comprising marker sequences.

Capture oligonucleotides typically comprise a nucleotide sequence capable of hybridizing or annealing to a nucleic acid fragment of interest (e.g. target fragment, reference fragment) or a segment thereof. A capture oligonucleotide may be naturally occurring or synthetic and may be DNA or RNA based. Capture oligonucleotides can allow for specific separation of, for example, a target and/or reference fragment away from other fragments in a nucleic acid sample. The term “specific” or “specificity”, as used herein, refers to the binding or hybridization of one molecule to another molecule, such as an oligonucleotide for a target polynucleotide. “Specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “capture oligonucleotide”, “capture oligo”, “oligo”, or “oligonucleotide” may be used interchangeably throughout the document, when referring to capture oligonucleotides. The following features of oligonucleotides can be applied to primers and other oligonucleotides, such as probes provided herein.

A capture oligonucleotide can be designed and synthesized using a suitable process, and may be of any length suitable for hybridizing to a nucleotide sequence of interest and performing separation and/or analysis processes described herein. Oligonucleotides may be designed based upon a nucleotide sequence of interest (e.g., target fragment sequence (e.g., marker sequence), reference fragment sequence). An oligonucleotide, in some embodiments, may be about 10 to about 300 nucleotides, about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. An oligonucleotide may be composed of naturally occurring and/or non-naturally occurring nucleotides (e.g., labeled nucleotides), or a mixture thereof. Oligonucleotides suitable for use with embodiments described herein, may be synthesized and labeled using known techniques. Oligonucleotides may be chemically synthesized according to the solid phase phosphoramidite triester method first described by Beaucage and Caruthers (1981) Tetrahedron Letts. 22:1859-1862, using an automated synthesizer, and/or as described in Needham-VanDevanter et al. (1984) Nucleic Acids Res. 12:6159-6168. Purification of oligonucleotides can be effected by native acrylamide gel electrophoresis or by anion-exchange high-performance liquid chromatography (HPLC), for example, as described in Pearson and Regnier (1983) J. Chrom. 255:137-149.

All or a segment of an oligonucleotide sequence (naturally occurring or synthetic) may be substantially complementary to a target and/or reference fragment sequence or segment thereof, in some embodiments. As referred to herein, “substantially complementary” with respect to sequences refers to nucleotide sequences that will hybridize with each other. The stringency of the hybridization conditions can be altered to tolerate varying amounts of sequence mismatch. Included are target/reference and oligonucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Oligonucleotides that are substantially complimentary to a nucleic acid sequence of interest (e.g., target fragment sequence (e.g., marker sequence), reference fragment sequence) or segment thereof are also substantially similar to the compliment of the target nucleic acid sequence or relevant segment thereof (e.g., substantially similar to the anti-sense strand of the nucleic acid). One test for determining whether two nucleotide sequences are substantially similar is to determine the percent of identical nucleotide sequences shared. As referred to herein, “substantially similar” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other.

Annealing conditions (e.g., hybridization conditions) can be determined and/or adjusted, depending on the characteristics of the oligonucleotides used in an assay. Oligonucleotide sequence and/or length sometimes may affect hybridization to a nucleic acid sequence of interest. Depending on the degree of mismatch between an oligonucleotide and nucleic acid of interest, low, medium or high stringency conditions may be used to effect the annealing. As used herein, the term “stringent conditions” refers to conditions for hybridization and washing. Methods for hybridization reaction temperature condition optimization are known in the art, and may be found in Current Protocols in Molecular Biology, John Wiley & Sons, N.Y., 6.3.1-6.3.6 (1989). Aqueous and non-aqueous methods are described in that reference and either can be used. Non-limiting examples of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C. Stringent hybridization temperatures can also be altered (i.e. lowered) with the addition of certain organic solvents, formamide for example. Organic solvents, like formamide, reduce the thermal stability of double-stranded polynucleotides, so that hybridization can be performed at lower temperatures, while still maintaining stringent conditions and extending the useful life of nucleic acids that may be heat labile.

As used herein, the “hybridizing” refers to annealing a first nucleic acid molecule to a second nucleic acid molecule under low, medium or high stringency conditions, or under nucleic acid synthesis conditions. Hybridizing can include instances where a first nucleic acid molecule anneals to a second nucleic acid molecule, where the first and second nucleic acid molecules are complementary. As used herein, “specifically hybridizes” refers to preferential hybridization under nucleic acid synthesis conditions of an oligonucleotide to a nucleic acid molecule having a sequence complementary to the oligonucleotide compared to hybridization to a nucleic acid molecule not having a complementary sequence. For example, specific hybridization includes the hybridization of a capture oligonucleotide to a target fragment sequence that is complementary to the oligonucleotide.

In some embodiments, one or more capture oligonucleotides are associated with an affinity ligand such as a member of a binding pair (e.g., biotin) or antigen that can bind to a capture agent such as avidin, streptavidin, an antibody, or a receptor. For example, a capture oligonucleotide may be biotinylated such that it can be captured onto a streptavidin-coated bead.

In some embodiments, one or more capture oligonucleotides and/or capture agents are effectively linked to a solid support or substrate. A solid support or substrate can be any physically separable solid to which a capture oligonucleotide can be directly or indirectly attached including, but not limited to, surfaces provided by arrays, microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, the solid phase can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe²⁺ and Fe³⁺). The oligonucleotides may be linked to the solid support by covalent bonds or by non-covalent interactions and may be linked to the solid support directly or indirectly (e.g., via an intermediary agent such as a spacer molecule or biotin). A capture oligonucleotide or probe may be linked to the solid support before, during or after nucleic acid capture.

### Systems, Machines and Components

Certain processes and methods described herein (e.g., detecting, quantifying, selecting, determining) sometimes cannot be performed without a computer, processor, software, module or other apparatus. Methods described herein often are computer-implemented methods, and one or more portions of a method sometimes are performed by one or more processors. In some embodiments, processes and methods described herein (e.g., quantifying, selecting, determining) are performed by automated methods. In some embodiments, an automated method is embodied in software, modules, processors, peripherals and/or an apparatus comprising the like, that detect, quantify, select and/or determine as described herein. As used herein, software refers to computer readable program instructions that, when executed by a processor, perform computer operations, as described herein.

Apparatus, software and interfaces may be used to conduct methods described herein. Using apparatus, software and interfaces, a user may enter, request, query or determine options for using particular information, programs or processes (e.g., detecting, quantifying, selecting, determining), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by a suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more apparatus. Each apparatus comprises one or more of memory, one or more processors, and instructions. Where a system includes two or more apparatus, some or all of the apparatus may be located at the same location, some or all of the apparatus may be located at different locations, all of the apparatus may be located at one location and/or all of the apparatus may be located at different locations. Where a system includes two or more apparatus, some or all of the apparatus may be located at the same location as a user, some or all of the apparatus may be located at a location different than a user, all of the apparatus may be located at the same location as the user, and/or all of the apparatus may be located at one or more locations different than the user.

A system sometimes comprises a computing apparatus and a sequencing apparatus, where the sequencing apparatus is configured to receive physical nucleic acid and generate data (e.g., sequence reads), and the computing apparatus is configured to process the data from the sequencing apparatus. The computing apparatus sometimes is configured to detect or quantify one or more markers, and/or make a determination as described herein.

When using a system or apparatus, a user may, for example, place a query to software which then may acquire a data set via internet access, and in certain embodiments, a programmable processor may be prompted to acquire a suitable data set based on given parameters. A programmable processor also may prompt a user to select one or more data set options selected by the processor based on given parameters. A programmable processor may prompt a user to select one or more data set options selected by the processor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, apparatus, or computer programs.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output means may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus may serve as data that can be input via an input device. In certain embodiments, mapped sequence reads may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system or apparatus may include software useful for performing a process described herein, and software can include one or more modules for performing such processes (e.g., sequencing module, logic processing module, data display organization module). The term “software” refers to computer readable program instructions that, when executed by a computer, perform computer operations. Instructions executable by the one or more processors sometimes are provided as executable code, that when executed, can cause one or more processors to implement a method described herein. A module described herein can exist as software, and instructions (e.g., processes, routines, subroutines) embodied in the software can be implemented or performed by a processor. For example, a module (e.g., a software module) can be a part of a program that performs a particular process or task. The term “module” refers to a self-contained functional unit that can be used in a larger apparatus or software system. A module can comprise a set of instructions for carrying out a function of the module. A module can transform data and/or information. Data and/or information can be in a suitable form. For example, data and/or information can be digital or analogue. In some cases, data and/or information can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, elevations, ranges, thresholds, signals, displays, representations, or transformations thereof. A module can accept or receive data and/or information, transform the data and/or information into a second form, and provide or transfer the second form to an apparatus, peripheral, component or another module. A processor can, in some instances, carry out the instructions in a module. In some embodiments, one or more processors are required to carry out instructions in a module or group of modules. A module can provide data and/or information to another module, apparatus or source and can receive data and/or information from another module, apparatus or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and processor capable of implementing instructions from a module can be located in an apparatus or in different apparatus. A module and/or processor capable of implementing an instruction for a module can be located in the same location as a user (e.g., local network) or in a different location from a user (e.g., remote network, cloud system). In embodiments in which a method is carried out in conjunction with two or more modules, the modules can be located in the same apparatus, one or more modules can be located in different apparatus in the same physical location, and one or more modules may be located in different apparatus in different physical locations.

An apparatus, in some embodiments, comprises at least one processor for carrying out the instructions in a module. Counts of sequence reads mapped to genomic sections of a reference genome sometimes are accessed by a processor that executes instructions configured to carry out a method described herein. Counts that are accessed by a processor can be within memory of a system, and the counts can be accessed and placed into the memory of the system after they are obtained. In some embodiments, an apparatus includes a processor (e.g., one or more processors) which processor can perform and/or implement one or more instructions (e.g., processes, routines and/or subroutines) from a module. In some embodiments, an apparatus includes multiple processors, such as processors coordinated and working in parallel. In some embodiments, an apparatus operates with one or more external processors (e.g., an internal or external network, server, storage device and/or storage network (e.g., a cloud)). In some embodiments, an apparatus comprises a module. In some embodiments, an apparatus comprises one or more modules. An apparatus comprising a module often can receive and transfer one or more of data and/or information to and from other modules. In some cases, an apparatus comprises peripherals and/or components. In some embodiments, an apparatus can comprise one or more peripherals or components that can transfer data and/or information to and from other modules, peripherals and/or components. In some embodiments, an apparatus interacts with a peripheral and/or component that provides data and/or information. In some embodiments, peripherals and components assist an apparatus in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a processor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash drives, RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational geometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of an identified result, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more processors in certain embodiments. A processor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A processor may implement software in a system. In some embodiments, a processor may be programmed to automatically perform a task described herein that a user could perform. Accordingly, a processor, or algorithm conducted by such a processor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

One or more entities can perform a process described herein, and an apparatus or system or computer program product can facilitate performance of the process. One entity can generate marker data, and utilize the maker data in a method, system, apparatus or computer program product described herein, in some embodiments. In certain embodiments, marker data are transferred by one entity to a second entity for use by the second entity in a method, system, apparatus or computer program product described herein. In some embodiments, one entity generates marker data and quantifies the marker, and transfers the marker quantification to a second entity that makes a determination described herein. In some embodiments, one entity obtains a biological sample from a subject, optionally isolates nucleic acid from the sample, and transfers the sample and/or nucleic acid to a second entity that generates marker data from the sample and/or nucleic acid.

Provided herein in certain aspects is a system comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined. Each of the one or more markers sometimes is a particular methylation state of a region of the nucleic acid, and the methylation state of each of the one or more markers sometimes is the same or substantially the same for a cell type in subjects having a medical condition and for the cell type in subjects not having the medical condition.

Also provided herein in certain aspects is an apparatus comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined. Each of the one or more markers sometimes is a particular methylation state of a region of the nucleic acid, and the methylation state of each of the one or more markers sometimes is the same or substantially the same for a cell type in subjects having a medical condition and for the cell type in subjects not having the medical condition.

Provided also in certain aspects is a computer program product tangibly embodied on a computer-readable medium, comprising instructions that when executed by one or more processors are configured to quantify the amount of each of one or more markers in nucleic acid from data pertaining to the one or more markers in the nucleic acid, wherein the presence or absence of a change in the methylation state of the one or more markers is not determined. Each of the one or more markers sometimes is a particular methylation state of a region of the nucleic acid, and the methylation state of each of the one or more markers sometimes is the same or substantially the same for a cell type in subjects having a medical condition and for the cell type in subjects not having the medical condition.

Provided herein in certain aspects is a system comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to select loci for which the methylation state is the same or substantially the same for a cell type in subjects having a medical condition and for the cell type in subjects not having the medical condition, whereby a collection of nucleic acid markers is prepared.

Provided herein in certain aspects is an apparatus comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to select loci for which the methylation state is the same or substantially the same for a cell type in subjects having a medical condition and for the cell type in subjects not having the medical condition, whereby a collection of nucleic acid markers is prepared.

Provided also in certain aspects is a computer program product tangibly embodied on a computer-readable medium, comprising instructions that when executed by one or more processors are configured to (a) determine the methylation state of multiple loci in nucleic acid from multiple cell types from multiple subjects; and (b) select loci for which the methylation state is the same or substantially the same for a cell type in subjects having a medical condition and for the cell type in subjects not having the medical condition, whereby a collection of nucleic acid markers is prepared.

Provided herein in certain aspects is a system comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to (a) select loci for which the methylation state is the same or substantially the same for a cell type in subjects having a medical condition and for the cell type in subjects not having the medical condition, and (b) design amplification primers, each of which primers is capable of amplifying each of the loci selected in (a), whereby a collection of amplification primers is obtained.

Provided herein in certain aspects is an apparatus comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to (a) select loci for which the methylation state is the same or substantially the same for a cell type in subjects having a medical condition and for the cell type in subjects not having the medical condition, and (b) design amplification primers, each of which primers is capable of amplifying each of the loci selected in (a), whereby a collection of amplification primers is obtained.

Provided also in certain aspects is a computer program product tangibly embodied on a computer-readable medium, comprising instructions that when executed by one or more processors are configured to (a) determine the methylation state of multiple loci in nucleic acid from multiple cell types from multiple subjects; (b) select loci for which the methylation state is the same or substantially the same for a cell type in subjects having a medical condition and for the cell type in subjects not having the medical condition; and (c) design amplification primers, each of which primers is capable of amplifying each of the loci selected in (b), whereby a collection of amplification primers is obtained.

Data pertaining to one or more markers utilized for the instructions is any suitable data that permits quantification of the one or more markers in the nucleic acid. Such data can be from a suitable detection or quantification platform technology, non-limiting examples of which technology include mass spectrometry, amplification (e.g., digital PCR, quantitative polymerase chain reaction (qPCR)), sequencing (e.g., nanopore sequencing, base extension sequencing (e.g., single base extension sequencing)), array hybridization (e.g., microarray hybridization; gene-chip analysis), flow cytometry, gel electrophoresis (e.g., capillary electrophoresis), cytofluorimetric analysis, fluorescence microscopy, confocal laser scanning microscopy, laser scanning cytometry, affinity chromatography, manual batch mode separation, electric field suspension, the like and combinations of the foregoing. Data sometimes includes marker data, and sometimes the methylation state of one or more nucleic acid loci.

In certain embodiments, a system, apparatus and/or computer program product comprises: (i) a sequencing module configured to obtain nucleic acid sequence reads; (ii) a mapping module configured to map nucleic acid sequence reads to portions of a reference genome; (iii) a weighting module configured to weight genomic sections; (iv) a filtering module configured to filter genomic sections or counts mapped to a genomic section; (v) a counting module configured to provide counts of nucleic acid sequence reads mapped to portions of a reference genome; (vi) a normalization module configured to provide normalized counts; (vii) a quantification module configured to quantify one or more markers in nucleic acid; (viii) a methylation state detection module configured to determine a particular methylation state of at least one of the one or more markers; (ix) a categorization module configured to determine whether a methylation state is maintained or different in different cell types; (x) a selection module configured to select one or markers meeting certain criteria; (xi) a plotting module configured to graph and display data; (xii) an outcome module configured to determine an outcome (e.g., outcome determinative of the presence or absence of a fetal aneuploidy) or a determination module configured to determine the presence or absence of a determination (e.g., determining the likelihood a test subject has a medical disorder or is predisposed to having the medical disorder; determining the presence or absence of a progression of a medical disorder in a test subject; determining the presence or absence of a response to a therapy administered to a test subject; determining whether a dosage of a therapeutic agent administered to a test subject having a medical condition should be increased, decreased or maintained); (xiii) a data organization module configured to receive, organize and/or display marker data for quantification; (xiv) a logic processing module configured to perform one or more of map sequence reads, count mapped sequence reads, normalize counts, quantify marker(s), compare markers and generate an outcome or determination; (xv) a marker comparison module configured to compare a marker quantification to another marker quantification or cutoff value; (xvi) primer design module configured to design primers for amplifying particular loci; (xvii) the like; or (xviii) combination of two or more of the foregoing.

In some embodiments a sequencing module and mapping module are configured to transfer sequence reads from the sequencing module to the mapping module. A mapping module and counting module sometimes are configured to transfer mapped sequence reads from the mapping module to the counting module. A counting module and filtering module sometimes are configured to transfer counts from the counting module to the filtering module. A counting module and weighting module sometimes are configured to transfer counts from the counting module to the weighting module. A mapping module and filtering module sometimes are configured to transfer mapped sequence reads from the mapping module to the filtering module. A mapping module and weighting module sometimes are configured to transfer mapped sequence reads from the mapping module to the weighting module. In some embodiments, a weighting module, filtering module and counting module are configured to transfer filtered and/or weighted genomic sections from the weighting module and filtering module to the counting module. A weighting module and normalization module sometimes are configured to transfer weighted genomic sections from the weighting module to the normalization module. A filtering module and normalization module sometimes are configured to transfer filtered genomic sections from the filtering module to the normalization module. A normalization module sometimes is configured to transfer mapped normalized sequence read counts to one or more of the comparison module, range setting module, categorization module, adjustment module, outcome module or plotting module. A quantification module sometimes is configured to receive marker data from a data organization module, and sometimes is configured to transmit data to a marker comparison module or determination module. A methylation state detection module sometimes is configured to receive data from a data organization module and sometimes is configured to transmit data to a quantification module. A categorization module sometimes is configured to receive data from a data organization module, quantification module or methylation state detection module, and sometimes is configured to transmit data to a selection module or determination module. A selection module sometimes is configured to receive data from a data organization module, methylation state module or quantification module, and sometimes is configured to transmit data to a quantification module, a categorization module, selection module or determination module. A primer design module sometimes is configured to receive data from a quantification module, selection module, marker comparison module and sometimes is configured to display polynucleotides for one or more designed primers.

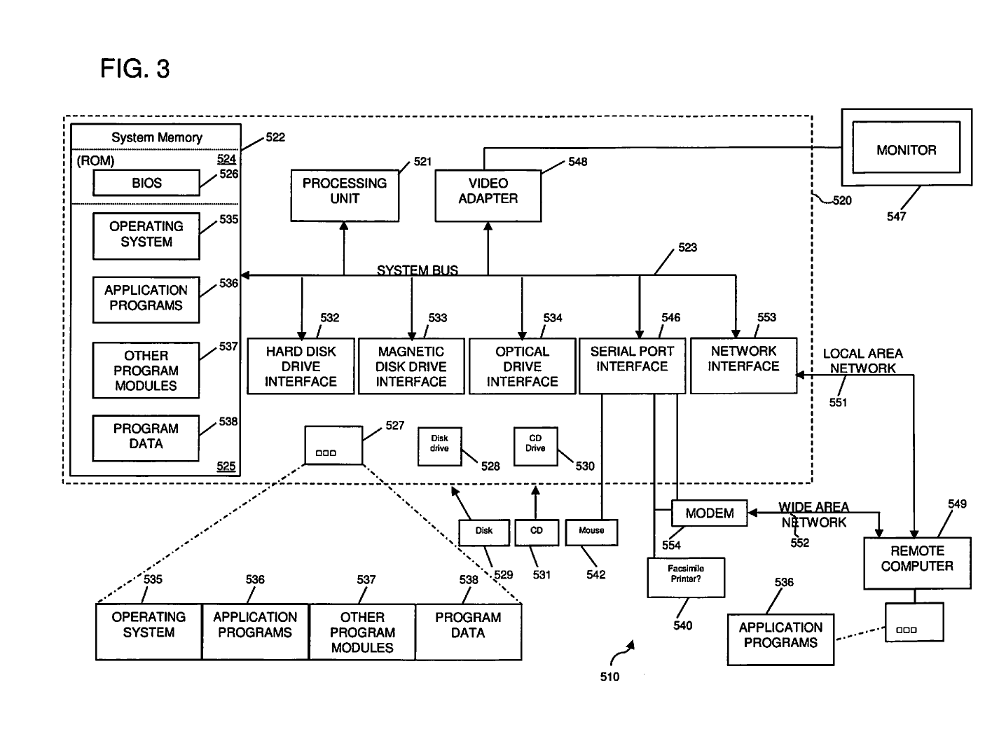

FIG. 3 illustrates a non-limiting example of a computing environment 510 in which various systems, methods, algorithms, and data structures described herein may be implemented. The computing environment 510 is only one example of a suitable computing environment and is not intended to suggest any limitation as to the scope of use or functionality of the systems, methods, and data structures described herein. Neither should computing environment 510 be interpreted as having any dependency or requirement relating to any one or combination of components illustrated in computing environment 510. A subset of systems, methods, and data structures shown in FIG. 3 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 510 of FIG. 3 includes a general purpose computing device in the form of a computer 520, including a processing unit 521, a system memory 522, and a system bus 523 that operatively couples various system components including the system memory 522 to the processing unit 521. There may be only one or there may be more than one processing unit 521, such that the processor of computer 520 includes a single central-processing unit (CPU), or a plurality of processing units, commonly referred to as a parallel processing environment. The computer 520 may be a conventional computer, a distributed computer, or any other type of computer.

The system bus 523 may be any of several types of bus structures including a memory bus or memory controller, a peripheral bus, and a local bus using any of a variety of bus architectures. The system memory may also be referred to as simply the memory, and includes read only memory (ROM) 524 and random access memory (RAM). A basic input/output system (BIOS) 526, containing the basic routines that help to transfer information between elements within the computer 520, such as during start-up, is stored in ROM 524. The computer 520 may further include a hard disk drive interface 527 for reading from and writing to a hard disk, not shown, a magnetic disk drive 528 for reading from or writing to a removable magnetic disk 529, and an optical disk drive 530 for reading from or writing to a removable optical disk 531 such as a CD ROM or other optical media.

The hard disk drive 527, magnetic disk drive 528, and optical disk drive 530 are connected to the system bus 523 by a hard disk drive interface 532, a magnetic disk drive interface 533, and an optical disk drive interface 534, respectively. The drives and their associated computer-readable media provide nonvolatile storage of computer-readable instructions, data structures, program modules and other data for the computer 520. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 529, optical disk 531, ROM 524, or RAM, including an operating system 535, one or more application programs 536, other program modules 537, and program data 538. A user may enter commands and information into the personal computer 520 through input devices such as a keyboard 540 and pointing device 542. Other input devices (not shown) may include a microphone, joystick, game pad, satellite dish, scanner, or the like. These and other input devices are often connected to the processing unit 521 through a serial port interface 546 that is coupled to the system bus, but may be connected by other interfaces, such as a parallel port, game port, or a universal serial bus (USB). A monitor 547 or other type of display device is also connected to the system bus 523 via an interface, such as a video adapter 548. In addition to the monitor, computers typically include other peripheral output devices (not shown), such as speakers and printers.

The computer 520 may operate in a networked environment using logical connections to one or more remote computers, such as remote computer 549. These logical connections may be achieved by a communication device coupled to or a part of the computer 520, or in other manners. The remote computer 549 may be another computer, a server, a router, a network PC, a client, a peer device or other common network node, and typically includes many or all of the elements described above relative to the computer 520, although only a memory storage device 550 has been illustrated in FIG. 3. The logical connections depicted in FIG. 3 include a local-area network (LAN) 551 and a wide-area network (WAN) 552. Such networking environments are commonplace in office networks, enterprise-wide computer networks, intranets and the Internet, which all are types of networks.

When used in a LAN-networking environment, the computer 520 is connected to the local network 551 through a network interface or adapter 553, which is one type of communications device. When used in a WAN-networking environment, the computer 520 often includes a modem 554, a type of communications device, or any other type of communications device for establishing communications over the wide area network 552. The modem 554, which may be internal or external, is connected to the system bus 523 via the serial port interface 546. In a networked environment, program modules depicted relative to the personal computer 520, or portions thereof, may be stored in the remote memory storage device. It is appreciated that the network connections shown are non-limiting examples and other communications devices for establishing a communications link between computers may be used.

### EXAMPLES

The examples set forth below illustrate certain embodiments and do not limit the technology.

### Example 1

### Marker Selection and Quantification

Provided in this example is a method that includes a primary target identification and characterization phase followed by application of those results to a discriminatory assay. For identification of targets, one or more of the following techniques is utilized (i) MeDIP coupled to microarray analysis, (ii) MeDIP coupled to sequencing analysis, (iii) MCIp coupled to microarray analysis, (iv) MCIp coupled to sequencing analysis, (v) whole genome bisulfite sequencing, (vi) reduced representation bisulfite sequencing, (vii) methylation sensitive polymerase chain reaction, (viii) sodium bisulfite coupled to MassCLEAVE technology, (ix) sodium bisulfite coupled to iPlex technology, or (x) methylation sensitive restriction enzyme differentiation between unmethylated and methylated oligonucleotide fragments. Using one or more than one of the aforementioned approaches, regions are identified that exhibit the same DNA methylation or other discriminatory pattern between a non-diseased and diseased sample from the same cell type, which together show a different DNA methylation or other discriminatory cell type, likely to be hematopoietic in origin. The output of such a process is identification of regions only methylated in a single cell type and invariant between the normal and diseased condition of that cell type.

For markers identified by such a process, nucleic acids present in plasma or serum is evaluated using one of the aforementioned techniques for differentiating DNA methylation. The absolute or relative quantity of the target nucleic acid is computed and compared to the standard range for a particular cell type. Each target cell type has a different target or set of targets used to estimate its abundance. Deviation of the absolute or relative quantification, which can be an increased amount relative to the established range, is classified as having an overabundance of the particular cell type.

Regions listed in the table below exhibit significant differential methylation (p<0.05; t-test) when comparing placenta to buffy coat (two distinct cell types) but not (p>0.05; t-test) when comparing euploid to trisomy 21 (T21) placenta (samples derived from same tissue showing similar methylation, independent of genetic condition). Methylation levels were measured using MassCLEAVE analysis (EpiTYPER) on a set of 6 buffy coat samples, 26 euploid placenta samples, and 6 trisomy 21 (T21) placenta samples. Chromosome start and end positions are according to the hg19 build of the human reference genome.

| | | | Buffy Coat | | Euploid Placenta | | T21 Placenta | | p.values (unpaired t test) | |
|---|---|---|---|---|---|---|---|---|---|---|
| chrom | start | end | Mean | Std Dev | Mean | Std Dev | Mean | Std Dev | EupVsT21 Placenta | PlacentaVsBuffy Coat |
| chr1 | 11252 | 11375 | 0.478 | 0.069 | 0.095 | 0.014 | 0.113 | 0.026 | 0.158831141 | 3.3035E−05 |
| chr1 | 1168057 | 1168519 | 0.791 | 0.016 | 0.469 | 0.083 | 0.527 | 0.088 | 0.19016707 | 2.29447E−15 |
| chr1 | 15392108 | 15392429 | 0.950 | 0.012 | 0.516 | 0.086 | 0.620 | 0.127 | 0.105537228 | 1.16762E−17 |
| chr1 | 18808947 | 18809092 | 0.882 | 0.046 | 0.528 | 0.073 | 0.663 | 0.144 | 0.071553899 | 3.22307E−09 |
| chr1 | 47905020 | 47905161 | 0.135 | 0.019 | 0.235 | 0.068 | 0.245 | 0.033 | 0.633670504 | 2.09043E−06 |
| chr1 | 50884703 | 50885193 | 0.086 | 0.018 | 0.130 | 0.029 | 0.157 | 0.052 | 0.271628651 | 0.000638998 |
| chr1 | 101005137 | 101005442 | 0.083 | 0.013 | 0.144 | 0.039 | 0.138 | 0.046 | 0.79060005 | 1.60455E−06 |
| chr1 | 110610236 | 110610524 | 0.141 | 0.014 | 0.268 | 0.057 | 0.326 | 0.068 | 0.096080705 | 8.15114E−10 |
| chr1 | 110611765 | 110612001 | 0.090 | 0.008 | 0.360 | 0.061 | 0.402 | 0.099 | 0.358989539 | 2.29199E−16 |
| chr1 | 110626239 | 110626463 | 0.087 | 0.009 | 0.225 | 0.051 | 0.270 | 0.079 | 0.234428053 | 8.08284E−12 |
| chr1 | 188726974 | 188727286 | 0.625 | 0.050 | 0.413 | 0.032 | 0.439 | 0.033 | 0.127132045 | 5.3125E−05 |
| chr1 | 228346101 | 228346318 | 0.862 | 0.027 | 0.301 | 0.066 | 0.362 | 0.079 | 0.124580764 | 4.4229E−19 |
| chr2 | 9879354 | 9879498 | 0.526 | 0.091 | 0.322 | 0.053 | 0.370 | 0.071 | 0.167107781 | 0.001976266 |
| chr2 | 30248594 | 30248837 | 0.704 | 0.047 | 0.258 | 0.064 | 0.314 | 0.058 | 0.071602254 | 1.58489E−09 |
| chr2 | 71205818 | 71206081 | 0.062 | 0.021 | 0.229 | 0.113 | 0.266 | 0.144 | 0.589534604 | 2.48495E−06 |
| chr2 | 74741410 | 74741696 | 0.058 | 0.018 | 0.140 | 0.053 | 0.175 | 0.072 | 0.30768704 | 4.4928E−06 |
| chr2 | 133012615 | 133012828 | 0.744 | 0.036 | 0.566 | 0.096 | 0.683 | 0.112 | 0.051631036 | 3.1512E−07 |
| chr2 | 219846451 | 219846776 | 0.127 | 0.037 | 0.208 | 0.047 | 0.200 | 0.050 | 0.751044501 | 0.001215032 |
| chr3 | 122745561 | 122745729 | 0.038 | 0.005 | 0.091 | 0.049 | 0.093 | 0.027 | 0.926975648 | 0.001499082 |

### Example 2

### Particular Embodiments

Provided hereafter is a listing of particular non-limiting embodiments of the technology.

A1. A method for quantifying one or more nucleic acid markers, comprising:
(a) exposing circulating cell-free nucleic acid to conditions that permit quantification of the amount of one or more markers in the nucleic acid, wherein:
   each of the one or more markers is a particular methylation state of a region of the nucleic acid, and
   the methylation state of each of the one or more markers is the same or substantially the same for a cell type in subjects having a medical condition and for the cell type in subjects not having the medical condition; and
(b) quantifying the amount of each of the one or more markers in the nucleic acid, thereby providing a quantification of the one or more markers,
with the proviso that the presence or absence of a change in the methylation state of the one or more markers is not determined.
   A1.1. The method of embodiment A1, wherein the methylation state of each of the one or more markers is the same for a cell type in subjects having a medical condition and for the cell type in subjects not having the medical condition.
   A2. The method of embodiment A1 or A1.1, wherein the methylation state of each of the one or more markers is specific for the cell type.
   A3. The method of any one of embodiments A1, A1.1 and A2, wherein (a) and (b) are performed for multiple markers in the nucleic acid.
   A4. The method of any one of embodiments A1 to A3, wherein the amount of each of the one or more markers is a copy number.
   A5. The method of any one of embodiments A1 to A4, comprising, prior to (a), determining the particular methylation state of each of the one or more markers for the cell type in subjects having the medical condition.
   A6. The method of any one of embodiments A1 to A5, comprising, prior to (a), determining the particular methylation state of each of the one or more markers for the cell type in subjects not having the medical condition.
   A7. The method of any one of embodiments A1 to A6, comprising, prior to (a), selecting markers for which each methylation state is the same or substantially the same for the cell type in subjects having a medical condition and for the cell type in subjects not having the medical condition.
   A8. The method of any one of embodiments A1 to A7, which comprises determining the likelihood the test subject has a medical disorder, or is pre-disposed to having the medical disorder, according to the quantification or relative quantification of the one or more markers in the nucleic acid.
   A9. The method of embodiment A8, wherein the medical disorder is the same or substantially the same as the medical condition.
   A10. The method of embodiment A9, wherein the medical disorder is not the same as the medical condition.
   A11. The method of any one of embodiments A8 to A10, wherein the medical disorder is a cell proliferative disorder, a wasting disorder, a degenerative disorder, an autoimmune disorder, pre-eclampsia, kidney disease, liver disease, acute toxicity, chronic toxicity, myocardial infarction or combination of the foregoing
   A12. The method of embodiment A11, wherein the medical disorder is a cell proliferative disorder.
   A13. The method of embodiment A11, wherein the medical disorder is a wasting disorder or degenerative disorder.
   A14. The method of embodiment A11, wherein the medical disorder is an autoimmune disorder.
   A15. The method of embodiment A11, wherein the medical disorder is pre-eclampsia.
   A16. The method of any one of embodiments A1 to A15, which comprises determining the presence or absence of a progression of a medical disorder in a test subject according to the quantification or relative quantification of the one or more markers.
   A17. The method of any one of embodiments A1 to A16, which comprises determining the presence or absence of a response to a therapy administered to a test subject according to the quantification of the one or more markers.
   A18. The method of any one of embodiments A1 to A17, which comprises determining whether the dosage of a therapeutic agent administered to a test subject having a medical disorder should be increased, decreased or maintained according to the quantification of the one or more markers.
   A19. The method of any one of embodiments A1 to A18, wherein the amount of at least one of the one or more markers increases in the circulating cell-free nucleic acid of the subjects having the medical condition.
   A20. The method of embodiment A19, wherein the amount of the at least one of the one or more markers increases by about 2-fold or more.
   A21. The method of embodiment A19 or A20, wherein the amount of the at least one of the one or more markers is not detectable in the circulating cell-free nucleic acid of the subjects not having the medical condition.
   A22. The method of any one of embodiments A19 to A21, wherein the amount of the at least one of the one or more markers in the circulating cell-free nucleic acid of the subjects not having the medical condition is about 20%, or less, of the total amount of the circulating cell-free nucleic acid.
   A23. The method of any one of embodiments A19 to A22, wherein the amount of the at least one of the one or more markers in the circulating cell-free nucleic acid of the subjects not having the medical condition is about 5-fold lower, or less, than the total amount of the circulating cell-free nucleic acid.
   A24. The method of any one of embodiments A1 to A18, wherein the amount of at least one of the one or more markers decreases in the circulating cell-free nucleic acid of the subjects having the medical condition.
   A25. The method of embodiment A24, wherein the amount of the at least one of the one or more markers decreases by about 2-fold or more.
   A26. The method of embodiment A24 or A25, wherein the amount of the at least one of the one or more markers is not detectable in the circulating cell-free nucleic acid of the subjects having the medical condition.
   A27. The method of any one of embodiments A24 to A26, wherein the amount of the at least one of the one or more markers in the circulating cell-free nucleic acid of the subjects not having the medical condition is about 80%, or more, of the total amount of the circulating cell-free nucleic acid.
   B1. A method for preparing a collection of nucleic acid markers, comprising:
(a) determining the methylation state of multiple loci in nucleic acid from multiple cell types from multiple subjects; and
(b) selecting loci for which the methylation state is the same or substantially the same for a cell type in subjects having a medical condition and for the cell type in subjects not having the medical condition; whereby a collection of nucleic acid markers is prepared.
   B2. The method of embodiment B1, which comprises synthesizing one or more loci in the collection of markers.
   B3. The method of embodiment B2, wherein the synthesizing comprises amplifying a portion of nucleic acid from a subject comprising one of the loci.
   C1. A method for obtaining a collection of amplification primers, comprising:
(a) determining the methylation state of multiple loci in nucleic acid from multiple cell types from multiple subjects;
(b) selecting loci for which the methylation state is the same or substantially the same for a cell type in subjects having a medical condition and for the cell type in subjects not having the medical condition; and
(c) designing amplification primers, each of which primers is capable of amplifying each of the loci selected in (b); whereby a collection of amplification primers is obtained.
   C2. The method of embodiment C2, which comprises synthesizing the collection of amplification primers.
   D1. A system comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined.
   D2. An apparatus comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined.
   D3. A computer program product tangibly embodied on a computer-readable medium, comprising instructions that when executed by one or more processors are configured to quantify the amount of each of one or more markers in nucleic acid from data pertaining to the one or more markers in the nucleic acid, wherein the presence or absence of a change in the methylation state of the one or more markers is not determined.
   E1. A system comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to select loci for which the methylation state is the same or substantially the same for a cell type in subjects having a medical condition and for the cell type in subjects not having the medical condition, whereby a collection of nucleic acid markers is prepared.
   E2. An apparatus comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to select loci for which the methylation state is the same or substantially the same for a cell type in subjects having a medical condition and for the cell type in subjects not having the medical condition, whereby a collection of nucleic acid markers is prepared.
   E3. A computer program product tangibly embodied on a computer-readable medium, comprising instructions that when executed by one or more processors are configured to (a) determine the methylation state of multiple loci in nucleic acid from multiple cell types from multiple subjects; and (b) select loci for which the methylation state is the same or substantially the same for a cell type in subjects having a medical condition and for the cell type in subjects not having the medical condition, whereby a collection of nucleic acid markers is prepared.
   F1. A system comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to (a) select loci for which the methylation state is the same or substantially the same for a cell type in subjects having a medical condition and for the cell type in subjects not having the medical condition, and (b) design amplification primers, each of which primers is capable of amplifying each of the loci selected in (a), whereby a collection of amplification primers is obtained.
   F2. An apparatus comprising one or more processors and memory, which memory comprises instructions executable by the one or more processors and which memory comprises data pertaining to the methylation state of multiple loci in nucleic acid from multiple cell types from multiple subjects; and which instructions executable by the one or more processors are configured to (a) select loci for which the methylation state is the same or substantially the same for a cell type in subjects having a medical condition and for the cell type in subjects not having the medical condition, and (b) design amplification primers, each of which primers is capable of amplifying each of the loci selected in (a), whereby a collection of amplification primers is obtained.
   F3. A computer program product tangibly embodied on a computer-readable medium, comprising instructions that when executed by one or more processors are configured to (a) determine the methylation state of multiple loci in nucleic acid from multiple cell types from multiple subjects; (b) select loci for which the methylation state is the same or substantially the same for a cell type in subjects having a medical condition and for the cell type in subjects not having the medical condition; and (c) design amplification primers, each of which primers is capable of amplifying each of the loci selected in (b), whereby a collection of amplification primers is obtained.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

What is claimed is:

1. A method for quantifying one or more nucleic acid markers, comprising:
(a) exposing circulating cell-free nucleic acid to conditions that permit quantification of the amount of one or more markers in the nucleic acid, wherein:
each of the one or more markers is a particular methylation state of a region of the nucleic acid, and
the methylation state of each of the one or more markers is the same or substantially the same for a cell type in subjects having a medical condition and for the cell type in subjects not having the medical condition; and
(b) quantifying the amount of each of the one or more markers in the nucleic acid, thereby providing a quantification of the one or more markers,
with the proviso that the presence or absence of a change in the methylation state of the one or more markers is not determined.

2. The method of claim 1, wherein the methylation state of each of the one or more markers is the same for a cell type in subjects having a medical condition and for the cell type in subjects not having the medical condition.

3. The method of claim 1, wherein the methylation state of each of the one or more markers is specific for the cell type.

4. The method of claim 1, wherein (a) and (b) are performed for multiple markers in the nucleic acid.

5. The method of claim 1, wherein the amount or relative amount of each of the one or more markers is a copy number.

6. The method of claim 1, comprising, prior to (a), determining a methylation state of each of the one or more markers for the cell type in subjects having the medical condition and for the cell type in subjects not having the medical condition.

7. The method of claim 6, comprising, prior to (a), selecting markers for which each methylation state is the same or substantially the same for the cell type in subjects having a medical condition and for the cell type in subjects not having the medical condition.

8. The method of claim 1, which comprises determining the likelihood the test subject has a medical disorder, or is pre-disposed to having the medical disorder, according to the quantification or relative quantification of the one or more markers in the nucleic acid.

9. The method of claim 8, wherein the medical disorder is the same or substantially the same as the medical condition.

10. The method of claim 8, wherein the medical disorder is not the same as the medical condition.

11. The method of claim 8, wherein the medical disorder is a cell proliferative disorder, a wasting disorder, a degenerative disorder, an autoimmune disorder, pre-eclampsia, kidney disease, liver disease, acute toxicity, chronic toxicity, myocardial infarction or combination of the foregoing.

12. The method of claim 1, which comprises determining the presence or absence of a progression of a medical disorder in a test subject according to the quantification of the one or more markers.

13. The method of claim 1, which comprises determining the presence or absence of a response to a therapy administered to a test subject according to the quantification of the one or more markers.

14. The method of claim 1, which comprises determining whether a dosage of a therapeutic agent administered to a test subject having a medical disorder should be increased, decreased or maintained according to the quantification of the one or more markers.

15. The method of claim 1, wherein the amount of at least one of the one or more markers increases in the circulating cell-free nucleic acid of the subjects having the medical condition.

16. The method of claim 15, wherein the amount of the at least one of the one or more markers increases by about 2-fold or more.

17. The method of claim 15, wherein the amount of the at least one of the one or more markers is not detectable in the circulating cell-free nucleic acid of the subjects not having the medical condition.

18. The method of claim 15, wherein the amount of the at least one of the one or more markers in the circulating cell-free nucleic acid of the subjects not having the medical condition is about 20%, or less, of the total amount of the circulating cell-free nucleic acid.

19. The method of claim 15, wherein the amount of the at least one of the one or more markers in the circulating cell-free nucleic acid of the subjects not having the medical condition is about 5-fold lower, or less, than the total amount of the circulating cell-free nucleic acid.

20. The method of claim 1, wherein the amount of at least one of the one or more markers decreases in the circulating cell-free nucleic acid of the subjects having the medical condition.

21. The method of claim 20, wherein the amount of the at least one of the one or more markers decreases by about 2-fold or more.

22. The method of claim 20, wherein the amount of the at least one of the one or more markers is not detectable in the circulating cell-free nucleic acid of the subjects having the medical condition.

23. The method of claim 20, wherein the amount of the at least one of the one or more markers in the circulating cell-free nucleic acid of the subjects not having the medical condition is about 80%, or more, of the total amount of the circulating cell-free nucleic acid.

24. A system comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined.

25. An apparatus comprising one or more processors and memory, which memory comprises instructions executable by the one or more processors and which memory comprises data pertaining to one or more markers in a nucleic acid; and which instructions executable by the one or more processors are configured to quantify the amount of each of the one or more markers in the nucleic acid from the data, wherein the presence or absence of a change in the methylation state of the one or more markers is not determined.

26. A computer program product tangibly embodied on a computer-readable medium, comprising instructions that when executed by one or more processors are configured to quantify the amount of each of one or more markers in nucleic acid from data pertaining to the one or more markers in the nucleic acid, wherein the presence or absence of a change in the methylation state of the one or more markers is not determined.
